SEQUENCE LISTING (19 sequences)

SEQ ID NO: 1 - DNA, 1503 bp, Artificial Sequence
  6S rRNA of the Bacillus simplex strain VMC 10/70
gacgaacgctggcggcgtgcctaatacatgcaagtcgagcgaatcgatgggagcttgctccctgagattagcggcggacgggtgagtaacacgtgggcaacctgcctataagactgggataacttcgggaaaccggagctaataccggatacgttcttttctcgcatgagagaagatggaaagacggtttacgctgtcacttatagatgggcccgcggcgcattagctagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgaagaaggccttcgggtcgtaaagttctgttgttagggaagaacaagtaccagagtaactgctggtaccttgacggtacctaaccagaaagccacggctaactacgtgccagcagccgcggtaatacgtaggtggcaagcgttgtccggaattattgggcgtaaagcgcgcgcaggtggttccttaagtctgatgtgaaagcccacggctcaaccgtggagggtcattggaaactggggaacttgagtgcagaagaggaaagtggaattccaagtgtagcggtgaaatgcgtagagatttggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggctttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaacctgcgaaggtaagcgaatcccataaagccattctcagttcggattgcaggctgcaactcgcctgcatgaagccggaatcgctagtaatcgcggatcagcatgccgcggtgaatacgttcccgggccttgtacacaccgcccgtcacaccacgagagtttgtaacacccgaagtcggtgaggtaaccttcatggagccagccgcctaaggtgggacagatgattggggtgaagtcgtaacaaggtagccgtatcggaagg

SEQ ID NO: 2 - DNA, 759 bp, Artificial Sequence
  gapA gene from the Bacillus simplex strain VMC 10/70
tatatgttagcgcaccttcttcaatacgatacaattcatggatcacttaatgaaaaagtaacagttgatggggattaccttgttgttgatggtcataaagtcaaagtattggctgaacgtgaccctgcacaattagcatggggtgaactaggagtagaagtagtagtagaatctacaggacgtttcacgaaacgtgcagatgcagctaagcatttagaagctggcgcgaaaaaagtaatcatctctgctcctgcatctgatgaagatatcacaatcgtcatgggtgtaaacgaagataaatatgatgcagctaaccaccatgtaatctctaatgcttcttgtacaacgaactgcttagctccatttgctaaagtgcttcacgaacaattcggaatcaaacgcggtatgatgactactgttcactcttatacaaatgatcagcaaatccttgatttgccacataaagattaccgccgtgcacgtgcagctgccgagaatatcattcctacaacaactggggcagcaaaagctgttgcacttgtccttcctgaacttaaagggaaattgaatggtatggcaatgcgcgtacctactccaaacgtgtctgttgtcgaccttgttgcagaacttgaaaaagacacaacagttgaagaagttaatgcagcattcaaaaaggcttctgaaggtgaattaaaaggaatccttgagtacagcgaacttccgctagtatcaactgactataacggtaacccatcatct

SEQ ID NO: 3 - DNA, 461 bp, Artificial Sequence
  pgk gene from the Bacillus simplex strain VMC 10/70
ctgcttcaactgaaggaatagcgcaccatctcccagccgttgccggcttgctgatggaaaaagagctttcagtacttggaaaagccctatccaacccagaacgtccttttacagctataattggcggagcaaaagtaaaggataagataggcgttatcgaaaaccttttggaaaaagtcgatcacttgatcattggtggtggattgggttatacattcattaaagcgcaaggtcatgaaatcggtaattctttattggaggaagacaaaatagaattggccaaatctttcatcgaaagtgcaaaagaaaaaggcgtaaaacttcatttgcctatcgatgcagtcgtaactgctgaattttcacctgatgcagagacggataatgtcgatattgatgctattccaaaggataaaatggctcttgatatcggaccaaaaacaagcgaattatttgcggatgta

SEQ ID NO: 4 - DNA, 1272 bp, Artificial Sequence
  uvrA gene from the Bacillus simplex strain VMC 10/70
catttccatagaccagaaaacgaccagtcgaaatcctcgttcaaccgtagggactgttacggagatctatgattatttaaggctgttatttgcacgggtcggcaggccgacctgtcccatccataatatagaaatcacctcacaaacgatagagcaaatggtggaccgcattcttgattaccctgagcgaaccaagcttcaagtattggcaccgctagtctcaggcagaaaagggacacatgcgaaagttttggaggaagttaagaaacaaggatatgtccgcattcgtgtgaacggggaaatgcatgacctcagcgatgagatcactctcgaaaaaaataaaaaacattcgattgaagtcatcatagaccggatcgtcataaaagagggtatcatggccaggcttgcagattcattggaaagtgctttgcagcttggcgaaggtaaagtcatcattgacgtcatgggtgaggaagagctgctgttcagtgaacatcatgcttgtccatactgcggattttcgattgaagagttagagcctagaatgttttccttcaatagcccgtttggggcctgcccggattgtgatggcttgggggcaaggcttgaggtggaccgtgatctggtcattccgaataacgaattaagcctccgtcaacatgcaattgcgccatgggagccgactagttctcaatattaccctcagctccttgaagcggtggcaaaccattatgggatagatatggatgtgtccgttaaggatttaccggaagagaagatggataaggtcttgcttggttcaggcaaagataagatatatttccgttataaaaatgattttggaagagtgcaagaaggatatattccttttgaaggagttttaagaaacatcgaaaggcgcttcaaggagacgagttctgactttattcgtgagcaaatgcagaaatacatgtcagaacatcactgtccaacctgtaagggtcatcgattgaaaaaagagagtctttccgtgctcattcaaggggtccatattagtgaaacgacagctttatcagtggaggatgcgttagtatttttcgatgagcttgatttgactgaaaaagaagctgcaattgcgagattgattttacgtgaaattcgtgagcggctcggtttcctggctaatgtaggtttggaatatttgacgttgagcagggcagcgggaactttatcaggcggtgaagcgcagcgtataagatggcgacgcagatcggttcccgattgactggagtc

SEQ ID NO: 5 - DNA, 1514 bp, Artificial Sequence
  16S rRNA of the Paenibacillus polymyxa strain VMC 10/96
gacgaacgctggcggcgtgcctaatacatgcaagtcgagcggggttgtgtagaagcttgcttctacataacctagcggcggacgggtgagtaacacgtaggcaacctgcccacaagacagggataactaccggaaacggtagctaatacccgatacatccttttcctgcatgggagaaggaggaaagacggagcaatctgtcacttgtggatgggcctgcggcgcattagctagttggtggggtaaaggcctaccaaggcgacgatgcgtagccgacctgagagggtgatcggccacactgggactgagacacggcccagactcctacgggaggcagcagtagggaatcttccgcaatgggcgaaagcctgacggagcaacgccgcgtgagtgatgaaggttttcggatcgtaaagctctgttgccagggaagaacgtcttgtagagtaactgctacaagagtgacggtacctgagaagaaagccccggctaactacgtgccagcagccgcggtaatacgtagggggcaagcgttgtccggaattattgggcgtaaagcgcgcgcaggcggctctttaagtctggtgtttaatccccgagggctcaactttcgggtcgcactggaaactggggagcttgagtgcagaagaggagagtggaattccacgtgtagcggtgaaatgcgtagagatgtggaggaacaccagtggcgaaggcgactctctgggctgtaactgacgctgaggcgcgaaagcgtggggagcaaacaggattagataccctggtagtccacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaaatcgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgccgaaggtggggtagatgattggggtgaagtcgtaacaaggtagccgtatcggaaggtgc

SEQ ID NO: 6 - DNA, 3505 bp, Artificial Sequence
  rpoB gene from the Paenibacillus polymyxa strain VMC 10/96
ttggcaggacatcttgttcaatatggtcgacgcactcggcgcagttatgcacgtattaatgaggtactcgaggttccgaacctgattgagatccaacaaaaatcctatgattggtttttggaggaaggattaagggaaatgtttcgggatatttctccaattcaggatttcactggaaatctgattctggagtttatcgactattctctcggagaacccaaatataccgttgacgacgcaaaggaacgcgacgttacgtatgcagcaccgcttcgggtaaaagtccggcttattaataaagaaaccggggaagtgaaagagcaggaagtattcatgggagacttcccgctgatgactgaaacgggtacgtttattattaacggtgcggaacgggttattgtcagccagttggttcgctctcccagcgtctatttcagcacaaaagtcgacaagaatgcgaaaacaacatacaccgcaacggtaattcctaaccggggggcttggctcgaactggagatggatgcgaaggatattatctatgtccggattgaccgtacccgtaaaattccggttacggtgttgctgcgtgcgctgggctttggcactgatgctgagattctggatttgctcggcaatgacgaatatatccgcaacacacttgataaagacaacacggattccaccgagaaagcgctgattgaaatttatgagcgtcttcgtccaggtgagccgcctacactggataacgcaaagagcttgctagttgctcgcttctttgatcctaaacgttatgatctggccaacgtaggccgttacaaaatcaataaaaagcttcacattaaaaaccgtttgttcaatcaacgacttgctgagactttgattgatacaacaactggtgaaatcatcgctgaagccggtcaaatggtagatcgccgcctgttggacgagattttggcccaactggaggaatcagtaggacatcgtacgtatcatgttgcgagtggtgtgctagaaagcaatgatattccacttcaaacaatcgatgtgttctcaccaattgaggatggtaaagtagtaaaactgattgctaacggaaatattgataaatcggttaagaatattacgcctgccgatattatttcctccatcagttattttattaacttgcttcacggaatcggaagtacggacgatattgaccatttgggtaaccgtcgtttgcgttctgtaggtgagttgctccaaaaccagttccgtatcggtttatcccgtatggaacgcgtagtgcgcgaaagaatgtcgattcaggatgctaatgtaattacgccacaggcattgattaacatacgtccagtaattgcttcgattaaagagttctttggtagctcgcagctgtctcagtttatggatcagacgaacccgcttgctgaattaacgcacaaacgtcgtctgtccgcactcggacccggcggtttgacgcgcgaacgcgcgggcatggaagtgcgtgacgtccatccgagtcactacggccgtatgtgtcctatcgagacaccagagggaccaaacattggtttgatcaactctttgtccacttttgcacgcattaacgagtatggatttatcgaagctccttatcgttgggtagatccaaaaaccggaaaagttacagaccagattgattacctgactgctgatgaagaagataactacattgtagctcaggcgaatgcggaattgacggaggaaaacacctttaaggatgaggttgtcattgtccgttataacaaacagtctgataacattattccgatggctagtagccgtgtcgattacatggacgtatcgcctaaacaggtcgtatcggtcgcgactgcactgattccgttcttggagaatgatgactctaaccgcgcattgatgggttccaacatgcagcgtcaggctgtcccgcttctgattccgaagtctccattggtcggaacaggaatggagcacaagtctgcaaaagattccggtgtttgcgttgtatccaaatacaacggagttatcgaacgttcttcggctaacgaaatttggttgcgtcgtatcgaaactgtagatggcgctgaagtgaagggcgacattgttaagtataaattacacaaatttatgcgatctaaccaaggaacttgcatcaaccaacgtccgattgtgaacagaggagatattgtcaaagttggcgatattcttgcggatggtccatctacagagatgggtgagttggcgttgggacgtaacgttgtcgttgccttcatgacttgggaaggttacaactacgaggatgcgatcttgctgagtgagaaactggttaaggaagatgtatacacctcgatccatattgaggaatacgaatccgaggctcgtgacacgaagcttggaccggaagaaatcactcgcgacattccaaatgtcggtgaagaagcgcttcgcaacttggatgagcgtggaatcatacgtattggtgctgaaattggcgcaggtgacattctcgttggtaaagtaacacctaaaggtgtgactgaattgacagctgaagaacgtctcttacacgcaatctttggtgagaaggcacgcgaggttcgcgatacttctttgagagttcctcacggaacagacgggattgttgtagatgtaaaggtatttacacgtgaaaatggcgatgaactgccaccaggtgtaaatcagttggttcgtgtatatattgctcaaaaacgtaaaatttccgaaggcgataaaatggctggacgtcacggtaacaagggtgtcgttgctcgtattttgcctgaagaagatatgccgttcctgccggatggcacaccagtacaggtcgttctgaacccgctgggcgtaccttcacggatgaacatcggacaggtgcttgaagtccatctgggtatggctgcaatgcgtcttggtattcatgtggcaactccagtattcgatggtgccaaggaatatgacgtattcgatacaatggaagaggcaggcatgcagcgtaatggtaagactgtgttgtatgacggacgtacgggtgatcgttttgaacgtgaagttactgtcggtgtcatgcacatgatcaaactggcgcacatggtcgatgataaaatccatgctcgttctacaggtccttactctctcgttacgcaacaaccattgggtggtaaagctcaattcggtggacagcgcttcggggagatggaagtatgggcattggaagcctacggtgcagcgtacacgcttcaggaaattttgactgtgaaatctgatgatgtggttggacgtgttaaaacttacgaatccattgtcaaaggtgaaaatgtacctgaaccgggtgttccagaatcatttaaggtcttgatcaaagagctgcaaagcttgggtatggacgtgaagattctgtctgaagacgaacaagagatcgaaatgagagagcttgatgatgaggatgacacaaccggcgataact

SEQ ID NO: 7 - DNA, 263 bp, Artificial Sequence
  nifH gene from the Paenibacillus polymyxa strain VMC 10/96
gctacatctggctgctgaaaggggcacggtagaggatttggagctggaggatgttgtccagaagggcttcggtgacattctgaacgtggaatgcggcgggccagagcctggtgtcggctgtgcaggacgcggcatcatcacagccattaattttctggaggaagagggggcctacgaagggctggattttgtttcctacgatgtactgggggacgtcgtgtgcgggggctttgccatgcccatccgcgaaaacaaggcccaga

SEQ ID NO: 8 - DNA, 20 bp, Artificial Sequence
  Forward (E8F)
agagtttgatcctggctcag

SEQ ID NO: 9 - DNA, 20 bp, Artificial Sequence
  Reverse (E1541R)
  misc_feature (15)..(15): n is a, c, g, or t
aaggaggtgatccanccrca

SEQ ID NO: 10 - DNA, 21 bp, Artificial Sequence
  Forward (gapA-F)
aacagatgctaatatgttagc

SEQ ID NO: 11 - DNA, 17 bp, Artificial Sequence
  Reverse (gapA-R)
gatttgtagaagatggg

SEQ ID NO: 12 - DNA, 20 bp, Artificial Sequence
  Forward (pgk-F)
caccgtgcacatgcttcaac

SEQ ID NO: 13 - DNA, 20 bp, Artificial Sequence
  Reverse (pgk-R)
ggacttttgattacatccgc

SEQ ID NO: 14 - DNA, 20 bp, Artificial Sequence
  Forward (uvrA-F)
ttaggccaagtggataaacc

SEQ ID NO: 15 - DNA, 20 bp, Artificial Sequence
  Reverse (uvrA-R)
gcatcgttatccctctgatg

SEQ ID NO: 16 - DNA, 18 bp, Artificial Sequence
  Forward (rpoB-1698f)
aacatcggtttgatcaac

SEQ ID NO: 17 - DNA, 20 bp, Artificial Sequence
  Reverse (rpoB-2041r)
cgttgcatgttggtacccat

SEQ ID NO: 18 - DNA, 29 bp, Artificial Sequence
  Forward (nifH-F)
ggctgcgatccvaaggccgaytcvacccg

SEQ ID NO: 19 - DNA, 30 bp, Artificial Sequence
  Reverse (nifH-R)
ctgvgccttgttytcgcggatsggcatggc

(19) United States
(12) Patent Application Publication
(10) Pub. No.: US 2021/0276927 A1
(43) Pub. Date: Sep. 9, 2021

(54) NOVEL PAENIBACILLUS POLYMYXA AND USES THEREOF

(71) Applicant: VALAGRO S.P.A., Atessa (IT)

(72) Inventors: Matteo DI MUZIO, Lanciano (IT); Ilaria LEBANO, Frosinone (IT); Juan Fernando MEJIA DE LOS RIOS, Lanciano (IT); Donata DI TOMMASO, Mozzagrogna (IT); Alberto PIAGGESI, Francavilla Al Mare (IT); Prem WARRIOR, Scottsdale, AZ (US)

(21) Appl. No.: 16/967,990

(22) PCT Filed: Feb. 7, 2018
(86) PCT No.: PCT/IB2018/050755

§ 371 Date: Aug. 6, 2020

(51) Int. Cl. C05F 11/08; C12N 1/20; C05F 5/00; C05F 11/02; C05F 1/00

(52) U.S. Cl. CPC C05F 11/08; C12N 1/205; C05F 1/007; C05F 11/02; C05F 5/002

(57) ABSTRACT

The present invention refers to new bacterial strains, a consortium comprising said strains, a composition comprising at least one of these novel bacterial strains, preferably in combination with a plant biostimulant and their use in agriculture, preferably, to improve nutrients up-take in plants and therefore to ameliorate plant growth and/or development.

### TECHNICAL FIELD

The present invention refers to new bacterial strains, a microbial consortium comprising said strains, and a composition comprising at least one of the novel bacterial strain, preferably in combination with a biostimulant for plants. Moreover, the present invention refers to the use of the new bacterial strains, the microbial consortium and the composition in agriculture, preferably to improve nutrients up-take in plants and therefore to ameliorate plant growth and/or development.

### BACKGROUND ART

In the past years, the heavy use of agro-chemicals and pesticides, which marked the First Green Revolution, strongly improved crop productivity, without taking in account adverse effects on environment and human health (Serpil, 2012), such as decreased soil fertility and increased susceptibility of plants to pests and disease (Jen-Hshuan Chen, 2006).

Nowadays, sustainable agriculture is considered as the new way to produce more food in a sustainable manner in order to meet the increasing demand for food of an ever-growing population, also counteracting the adverse effects of the climate change on crop productivity.

Biofertilizers have been considered a good and eco-friendly solution for sustainable agriculture compared to chemical fertilizers. Indeed, biofertilizers can significantly improve crop productivity, ameliorate nutrient up-take and make plants more tolerant to several biotic and abiotic stress in a sustainable manner (Deepak et al., 2014).

In this context, the present invention proposes novel bacteria capable of increasing solubilization in the soil of main macronutrients and/or micronutrients, such as phosphorus, zinc and iron. Therefore, these bacteria are useful to improve micro and/or macronutrient's uptake in plants and to ameliorate plant biology, in particular, the growth and/or the development of plants.

### SUMMARY OF THE INVENTION

A first aspect of the present invention refers to an isolated bacterial strain of genus Paenibacillus, species polymyxa said strain being deposited at the DSMZ with the accession number DSM32460 and the following name (denomination): Paenibacillus polymyxa strain VMC10/96. The strain is preferably characterized by at least one species-specific sequence selected from: SEQ ID NO: 5-7 or sequences having at least 80-99% identity wherein SEQ ID NO: 5 belongs to 16S rRNA gene sequence, SEQ ID NO: 6 belongs to rpoB gene sequence, and SEQ ID NO: 7 belongs to nifH gene sequence.

Moreover, said strain is preferably able to use at least one of the carbon source selected from: glycerol, arabinose, ribose, xylose, galactose, glucose, fructose, mannose, mannitol, methyl-aD-glucopyranoside, amygdalin, arbutin, esculin, salicin, cellobiose, maltose, lactose, melibiose, saccharose, trehalose, inulin, raffinose, amidon, glycogen, gentiobiose, turanose, potassium gluconate and any combination thereof; and/or it is positive for: beta-galactosidase, and/or the acetoin production, and/or gelatinase and/or the reduction of nitrates to nitrites; and/or it is resistant to at least one of the antibiotic agent, preferably present in a concentration ranging from 4-32 μg/ml, selected from: cefonicid, ceftazidime, ceftriaxone, ciprofloxacin, miokamycin, co-trimoxazole, lincomycin and any combination therefore; and/or susceptible to at least one of the antibiotic agent, preferably present in a concentration ranging from 4-200 μg/ml, selected from: netilimicin, tobramycin, amoxicillin, ampicillin, pefloxacin, azithromycin, roxitromycin, fosfomycin, rifampicin, and any combination thereof.

Preferably, the isolated bacterial strain of the invention is characterized by a population doubling of 37-50 minutes, moreover it may be used in a form selected from: fresh bacteria, frozen bacteria, dry bacteria, lyophilized bacteria, liquid suspension of bacteria, encapsulated bacteria in the form of spores, living bacteria, culture medium, preferably whole culture medium comprising the bacteria or bacteria free medium, extract of bacteria, preferably cell free extract, supernatant, lysate of bacteria, fraction of bacteria and metabolites derived from said bacteria.

Preferably, the strain of the invention may be mutated and/or edited.

According to a preferred embodiment, the strain of the invention is used in combination with further microorganisms, preferably selected from: bacteria, preferably PGPR or rhizobacteria, yeasts, mycorrhizae, fungi, any derivatives as disclosed above and any combination thereof. Preferably, the PGPR is selected from: Aeromonas rivuli, Agromyces fucosus, Bacillus spp. Bacillus mycoides, Bacillus licheniformis, Bacillus subtilis, Bacillus megaterium, Bacillus pumilus, Bacillus safensis, Microbacterium sp., Nocardia globerula, Stenotrophomonas spp., Pseudomonas spp, Pseudomonas fluorescens, Pseudomonas fulva, Pseudoxanthomonas dajeonensis, Rhodococcus coprophilus, Sphingopyxis macrogoltabida, Streptomyces spp., Enterobacter spp., Azotobacter spp., Azospiriullum spp., Rhizobium spp., Herbaspirillum spp., Lactobacillus spp., Lactobacillus acidophilus, Lactobacillus buchneri, Lactobacillus delbrueckii, Lactobacillus johnsonii, Lactobacillus murinus, Lactobacillus paraplantarum, Lactobacillus pentosus, Lactobacillus plantarum, Lactococcus tactis, and combinations thereof; and/or the yeast is selected from: Candida spp., Candida tropicalis, Saccharomyces spp., Saccharomyces bayanus, Saccharomyces boulardii, Saccharomyces cerevisiae, Saccharomyces exiguous, Saccharomyces pastorianus, Saccharomyces pombe, and combinations thereof; and/or the mycorrhiza is selected from: Glomus spp., Rhizophagus spp., Septoglomus spp., Funneliformis spp., and combinations thereof; and/or the fungus is selected from: Trichoderma spp., Trichoderma atroviride, Trichoderma viride, Trichoderma afroharzianum, Paecilomyces spp., Beauveria bassiana, Metarhizium spp., Lecanicillium lecanii, Penicillium spp., Aspergillus spp., Conythyrium minitans, Pythium spp, and combinations thereof. According to a preferred embodiment, the strain of the invention is used in combination with the Bacillus simplex strain deposited at the DSMZ with the accession number DSM32459 and having the following name (denomination): Bacillus simplex strain VMC10/70. Preferably the ratio between the bacteria, more preferably between Bacillus simplex strain VMC10/70 and Paenibacillus polymyxa strain VMC10/96 ranges from 1:10 to 1:100, preferably it is 1:1.

According to a preferred embodiment, the strain of the invention is used as living and/or dead and/or killed cells and/or as spores or as a fresh or frozen sample or dry, lyophilized or in liquid suspension, or encapsulated in the form of living and/or dead and/or killed cells and/or as spores.

According to a preferred embodiment, the strain of the invention is used in combination with a biostimulant, preferably a biostimulant used in agriculture, preferably said plant biostimulant comprises at least one of the following ingredients: an extract of algae, and/or an extract of microalgae and/or an extract of plant and/or a humic acid and/or a fulvic acid and/or animal byproducts. Preferably, said algae are brown algae, preferably seaweeds, more preferably said algae are selected from: Ascophyllum nodosum, Ecklonia maxima, Laminaria saccharina, Laminaria digitata, Fucus spiralis, Fucus serratus, F. vesiculosus, Macrocystis spp., Pelvetia canaliculata, Himantalia elongata, Undaria pinnatifida, Sargassum spp, and combinations thereof; said microalgae are selected from: Spirulina, Scenedesmus, Nannochloropsis, Haematococcus, Chlorella, Phaeodactylum, Arthrospyra, Tetraselmis, lsochrysis, Synechocystis, Clamydomonas, Parietochloris, Desmodesmus, Neochloris, Dunaliella, Thalassiosira, Pavlova, Navicula, Chaetocerous, and combinations thereof. Preferably said plant is selected from: beet, sugar cane, alfalfa, maize, brassica, halophytes, soya, wheat, yucca, quillaja, hop, coffee, citrus, olive, and combinations thereof.

Preferably said part of a plant is selected from: leaves, roots, stems, fruits, flowers, seeds, seedlings, bark, berries, skins, and combinations thereof.

Preferably the extraction process from plants or from algae or from microalgae comprises the following steps: (i) preparing a sample of algae and/or a sample of microalgae and/or a sample of plants; and (ii) placing the said sample(s) in contact with an aqueous solution comprising an extraction agent preferably a base and/or an acid and/or an enzyme, wherein the base is preferable an inorganic base, preferably selected from: NaOH, KOH, Na₂CO₃, K₂CO₃, NH₃, salts thereof, and any combination thereof; the acid is preferably selected from: H₂SO₄, HNO₃, HCl, H₃PO₄, and various acids of organic nature preferably selected from: acetic acid, citric acid, formic acid, butyric acid and ascorbic acid, gluconic acid, and any combination thereof; the enzyme is preferably selected from: papain, trypsin, amylase, pepsin, bromelain and specific enzymes that degrade organic polymers present in the algae, preferably alginases, and any combination thereof. Preferably the temperature of the extraction process ranges between −20 and 120° C., more preferably between 20 and 100° C.; and/or the extraction time ranges from a few minutes to several hours, more preferably between 30 minutes and 18 hours; and/or the extraction process is realised at atmospheric pressure or at a pressure up to 10 Bar, more preferably at a pressure ranging from 1 to 8 Bar. According to a preferred embodiment, the concentration of the extract from algae and/or microalgae and/or plant in the biostimulant ranges from 1 to 60%, preferably it ranges from 5 to 50%, more preferably from 10 to 20%, still more preferably around 15%; and/or the concentration of the humic acid in the biostimulant ranges from 1 to 20%; and/or the concentration of the fulvic acid in the biostimulant ranges from 1 to 20%, preferably from 5 to 10%. Preferably the biostimulant is present in a concentration ranging from 5 to 50%, preferably from 10 to 40%, more preferably from 15 to 25%, preferably around 20% and/or the bacteria are used in a concentration ranging from 0.01 to 10%, preferably from 0.05 to 5%, more preferably from 0.1 to 1%.

A further aspect of the present invention refers to a medium obtained/obtainable by culturing the isolated bacterial strain of the invention, wherein said medium comprises the cultured bacteria or is a bacteria-free medium.

A further aspect of the present invention refers to an extract, preferably bacteria-free extract, or a supernatant or a lysate or a fraction or a metabolite obtained/obtainable by culturing the strain of the invention.

A further aspect of the present invention refers to a composition, preferably an agricultural composition comprising the strain of the invention, and/or the medium of the invention; and/or the extract of the invention and a carrier, preferably an agricultural compatible carrier.

Preferably the strain, the medium, the extract or the composition of the invention is formulated as water-soluble concentrates, dispersable concentrates, emulsifiable concentrates, emulsions, suspensions, microemulsion, gel, microcapsules, granules, ultralow volume liquid, wetting powder, dustable powder, or seed coating formulations.

A further aspect of the present invention refers to the use of the strain, the medium, the extract or the composition of the invention in agriculture, preferably for increasing the availability, preferably in the soil, of nutrients and/or macro-micronutrients, preferably selected from: nitrogen, phosphorus, potassium, calcium, magnesium, sulfur, boron (B), copper, iron, manganese, molybdenum, zinc and combination thereof, preferably selected from phosphorus and/or zinc and/or iron. Preferably said phosphorus is any inorganic source of phosphorus, preferably any inorganic source of phosphate, more preferably selected from: tricalcium phosphate, ferric phosphate and aluminum phosphate; and/or said zinc is any inorganic source of zinc, more preferably selected from: zinc oxide, zinc(II) carbonate, zinc(II) phosphate; and/or said iron is as iron ions (Fe³⁺), preferably chelated by siderophores obtained/obtainable by the strain of the invention.

A further aspect of the present invention refers to the use of the strain, the medium, the extract or the composition of the invention for improving plant uptake, preferably from soil, and/or of improving plant growth.

Preferably the plant is a vegetable plant, preferably, the plant can be selected from: Solanaceae, Cucurbitaceae, Graminaceae (Poaceae), Pomaceae, Chenopodiaceae, Brassicaeae, Compositae, Liliaceae, Leguminosae, Rosaceae, Vitaceae, Rutaceae, Oleaceae, Moraceae, Malvaceae, Musaceae, Lauraceae, Anacardiaceae, Juglandaceae, Zingiberaceae, Labiateae, Piperaceae, Cannabaceae, Arecaceae, Punicaceae, Bromeliaceae, Rubiaceae, Theaceae, Caricaceae, Passifloraceae, Asteraceae, Actinidiaceae, Fagaceae, Fabaceae, Ginkoaceae, Simondsiaceae and combinations thereof. More preferably said plant is selected from: tomato, melon, eggplant, pepper, cucumber, zucchini, potato, cauliflower, onion, lettuce, spinach, cabbage, savaoy cabbage, corn, wheat, barley, soybean, peach, apricot, plum, apple, pear, strawberry, grapes, cotton, almonds, various ornamentals, and combinations thereof.

A further aspect of the present invention refers to a method of increasing the availability, preferably in the soil, of nutrients and/or macro-micronutrients, preferably selected from: nitrogen, phosphorus, potassium, calcium, magnesium, sulfur, boron (B), copper, iron, manganese, molybdenum, zinc and combination thereof, preferably selected from phosphorus and/or zinc and/or iron and/or of improving plant uptake, preferably from soil, and/or of improving plant growth, said method comprising at least one step of introducing into the soil at least one inoculum the strain, the medium, the extract or the composition of the invention. The method preferably comprises at least one further step of adding to the soil a source of phosphorus and/or zinc and/or iron to the soil. Preferably said phosphorus is any inorganic source of phosphorus, preferably any inorganic source of phosphate, more preferably selected from: tricalcium phosphate, ferric phosphate and aluminum phosphate; and/or said zinc is any inorganic source of zinc, more preferably selected from: zinc oxide, zinc(II) carbonate, zinc(II) phosphate; and/or said iron is as iron ions (Fe³⁺), preferably chelated by siderophores obtained/obtainable by the strain of the invention.

### BRIEF DESCRIPTION OF DRAWINGS

FIG. 1 shows protein mass spectra (in the range from 2 to about 12 KDa, peaks of a spectrum m/z values with a given intensity) of the bacterial strains of the invention (VMC 10/70 and VMC 10/96). Each peak in the graphs represents a different protein expressed by the microorganism and the intensity of the peaks represents the concentration of that proteins within the microbial cell. Since proteins are a direct expression of genome and genome of each strains is unique, thus the proteomic profile of each strain is unique and can be used as fingerprint differentiation and classification.

FIG. 2 shows the phylogenetic tree obtained from the alignment of the full 16 rRNA gene sequences, with the “Maximum Likelihood” statistical method, “Tamura-Nei” substitution model and “Complete Deletion” mode (No. of bootstrap replications: 1000). A. The strain Bacillus simplex VMC10/70 is related to the species B. simplex and Bacillus muralis as it shares 99.66% and 99.60% of sequence similarity with the 16S rRNA sequence of the corresponding Type Strains.

B. The strain Paenibacillus polymyxa VMC10/96 is phylogenetically related to the species Paenibacillus peoriae, within the P. polymyxa group, which includes Paenibacillus jamilae, Paenibacillus brasilensis, Paenibacillus kribensis and Paenibacillus terrae, besides P. polymyxa.

FIG. 3-A shows the P-solubilization index of the isolated phosphate solubilizing bacteria ranged from 1.05 to 1.41 at seven days of incubation at 30° C. In particular, Paenibacillus polymyxa VMC10/96 is the most efficient phosphate solubilizer (SI=1.41) followed by the type strain ATCC 842 (P. polymyxa−SI=1.39) and Bacillus simplex VMC10/70 (SI=1.12), whereas the smallest SI of 1.05 was detected from the commercial strain Bacillus subtilis QST713.

FIG. 3-B shows the solubilized P concentrations recorded among the bacterial strains after 5 days of incubationin culture medium supplemented with (Ca₃(PO₄)₂). The highest mobilized phosphate value (185 mg/L) is recorded from isolate Paenibacillus polymyxa VMC10/96 whereas the minimum concentration of soluble-P (118 mg/L) is observed in the cultures of Bacillus Simplex VMC10/70 on day 5 of incubation.

FIG. 4-A shows the Solubilization Efficiency (SE) revealed that, among the screened isolates, Bacillus simplex VMC10/70 and Paenibacillus polymyxa VMC10/96 are the best zinc solubilizer with SE=143.65 and 135.71 respectively, whereas Paenibacillus polymyxa ATCC 842 and Bacillus amyloliquefaciens ATCC BAA-390 are found to be unable to solubilize zinc oxide.

FIG. 4-B shows the highly significant (p<0.05) variation of solubilized Zn concentrations that is recorded among the bacterial strains in 3 days of incubation. The highest dissolved zinc values are recorded from isolate Bacillus Simplex VMC10/70 and Paenibacillus polymyxa VMC10/96 (8.87 and 8.40 mg/I respectively), whereas the other strain is definitely unable to mobilize zinc from its insoluble form.

FIG. 5 shows the production of siderophores by Bacillus simplex strain VMC10/70. The production of organic chelates was evaluated in an iron-free medium, seeking the induction of siderophore production. The strain Bacillus simplex VMC10/70 shows excellent results obtained on CAS agar medium plates. After evaluating the ability of each strain to release siderophores into culture media, Bacillus simplex strain VMC10/70 is able to produce the highest amounts of siderophores within 72 hours followed by the Bacillus subtilis QST713 and the type strain P. polymyxa strain ATCC 842, according to the halo diameter 5.1 cm, 1.5 cm and 1.3 respectively.

FIG. 6 shows that, after 24 hours of incubation the strains produce the largest amount of Endoglucanase according to the CMC degradation Index (DI). In particular, Bacillus simplex strain VMC10/70 shows the best endoglucanase producer among all bacteria with a DI=1.65, followed by Paenibacillus polymyxa VMC10/96 with a DI=1.63, whereas the smallest DI of 1.27 was detected from the commercial strain Bacillus subtilis QST713.

### DETAILED DESCRIPTION OF PREFERRED EMBODIMENTS OF THE INVENTION

The present invention refers to novel strains of bacteria isolated and characterized for the ability to solubilize macro and/or micro-nutrients that are essential, in general, for plant biology and, preferably, for plant growth.

As used herein, “strain” refers to isolate or a group of isolates exhibiting phenotypic, physiologic, metabolic and/or genotypic traits belonging to the same lineage and different/distinct from that of other strains belonging to the same species.

As used herein, “isolate” means a microorganism which has been removed from its original environment, including, but not limited to, soil, air, fresh water, sea water, algae, higher plants, seeds, roots, leaves, fruits, etc. Preferably, the “isolate” has to be “pure”, meaning that it does not comprise other microbial contaminants in the isolate, according to the isolation method reported in the present invention.

As used herein, “macronutrients or main nutrients” refer—in general—to any nutrient that plays an essential role in any key physical, physiological and biochemical process of the plant, providing the heathy and balanced growth and development of said plant during all its lifecycle. Preferably, said nutrient is selected from: nitrogen, phosphorus, potassium (also known as NPK elements), calcium, magnesium, sulfur and combinations thereof. Phosphorus (P) is one of the most indispensable macronutrients next to nitrogen for the growth and/or the development of plants. A greater part of soil phosphorus, around 95-99%, is present in insoluble form complexed with cations like iron, aluminum, and calcium, all of them being chemical forms of unavailable P that, therefore, cannot be utilized by the plants. The use of natural phosphate-bearing materials, such as rock phosphate (RP), as fertilizer for P-deficient soils has received due attention in recent years since substantial deposits of cheaper and low-grade RP are locally available in many countries of the world. However, the solubilization of natural phosphate-bearing materials, such as rock phosphate (RP), rarely occurs in nonacidic soils with a pH greater than 5.5 to 6.0. Conventionally, RP is chemically processed by reacting with sulfuric acid or phosphoric acid to produce partially acidulated RP. The process incurs high cost and makes the environmental health worse. A much cheaper and convenient alternative is reclamation of exhausted soil through use of P-solubilizing microorganisms that have opened the possibility for solubilization of RP in soils. In this scenario, soil microorganisms play a critical role in natural phosphorus cycle and recently microbial-based approaches have been proposed to improve the agronomic value of RP. Therefore, microbial-based products represent cheaper approaches compared to the higher cost of manufacturing phosphate fertilizer in industry and, at the same time, they avoid the environment pollution posed by a traditional chemical process.

Deficiency or excess of the main nutrients have relevant consequences such as great unbalance of the crops, delay of the growth, leaves and/or flowers loss, reduced photosynthetic activity, with subsequent low fruit production and, overall, the crops productivity is heavily reduced. In some cases, pathological consequences, such as plant tissues and/or fruit necrosis, can also take place.

As used herein, “plant biology” refers to any physiological and/or pathological plant process, preferably selected from: growth and productivity of plants, comprising plant biomass (mainly N, but also S), plants metabolism and energy production, flowers, fruit and seeds development (mainly P, but also Ca and Mg), efficiency of the photosynthetic process, osmotic balance, and/or production and quality of the fruit (mainly K, but also Ca).

As used herein, “micronutrients” relate to the main nutrients absorbed in very small amounts grams per hectare (g/ha) compared with macronutrients (kg/ha). Preferably, said micronutrient is selected from: boron (B), copper (Cu), iron (Fe), manganese (Mn), molybdenum (Mo), zinc (Zn) and combinations thereof. Zinc is an essential micronutrient, which means it is essential for plant growth and development, but is required in very small quantities. It is crucial to plant development, as it plays a significant part in a wide range of processes, such as enzymes activation that are responsible for the synthesis of certain proteins. It is used in the formation of chlorophyll and some carbohydrates, conversion of starches to sugars and its presence in plant tissue helps the plant to withstand cold temperatures. Zinc is essential in the growth hormone production and internode elongation. Zinc deficiency is probably the most common micronutrient deficiency in crops worldwide. Symptoms caused by zinc deficiency vary depending on the crop. Typically, they include one or some of the following: stunting-reduced height, interveinal chlorosis, brown spots on upper leaves and distorted leaves. Nowadays, zinc fertilizers application to zinc-deficient soil results to be the current approach to improve zinc availability. The most common fertilizer sources of zinc are zinc chelates (contain approximately 14% zinc), zinc sulfate (25-36% zinc) and zinc oxide (70-80% Zinc), where zinc sulfate is the most commonly used source of zinc. The major problem of the application of these fertilizers is that in most of the cases only a small part of the total applied Zn is available to plants while remaining gets fixed into soil. Thus, an alternative solution is to use zinc solubilizing bacteria that are able to solubilize insoluble sources of zinc, preferably Zinc Oxide-ZnO, Zinc(II) Carbonate —ZnCO₃, or Zinc(II) Phosphate-Zn₃(PO₄)₂.

Micronutrient deficiency affects crop yield and quality. While excess of some micronutrients, despite taking place in rare situation, can interfere with the uptake of other nutrients resulting in unbalanced plant development.

As used herein, “solubilization of nutrients and/or micro-macronutrients” refers to the process allowing the conversion of the mineral forms of macro/micro-elements, which are present in the soil under unavailable forms for plants, into forms which can dissolve in water, so that the plants can uptake them through their roots.

Advantageously, the bacterial strains of the invention are able to solubilize, preferably in the soil, macro/micro-nutrients, preferably starting from the inorganic forms of macro/micro-nutrients. In other words, the bacterial strains of the invention are able to improve the solubilization and/or the dissolution of macro/micro-nutrients, preferably in the soil, and, consequently, the bacterial strains of the invention are able to improve the availability, preferably in the soil, of micro and/or macronutrients as defined above for plants.

As used herein, “plant growth” refers to the extension and/or expansion of plant tissues and/or organs, which bring to an increase of the vegetative biomass. An optimal plant growth results in balanced development of the whole plant and boosts processes of differentiation in flowers, fruits, and seeds.

The novel strain of bacteria of the invention have been deposited with the International Depositary Authority: Deutsche Sammlung von Mikroorganismen and Zellkulturen GmbH (hereinafter DSMZ) according to the provisions of Budapest Treaty.

A first isolated bacterial strain of the invention is a member of the genus Bacillus, species simplex deposited on Mar. 15 2017 at the DSMZ with the accession number DSM32459 and the following name (denomination): Bacillus simplex strain VMC10/70. The Bacillus simplex strain of the invention is gram positive and is preferably characterized by rod-shaped cells. The colony—when the bacteria are grown on nutrient agar—shows a cream color and is characterized by a size having an average diameter of 2-10 millimeters, preferably 3-6 millimeters.

Preferably, the colony's shape of Bacillus simplex strain is irregular, slightly raised and/or umbonate. According to a preferred embodiment of the invention, the Bacillus simplex strain of the invention is able to grow at a temperature ranging from less than 4 up to 40° C.

According to a further preferred embodiment of the invention, the Bacillus simplex strain of the invention is able to use at least one of the carbon source selected from: arabinose, glucose, fructose, mannose, mannitol, malate, N-acetylglucosamine, maltose, saccharose, trehalose, fucose, potassium gluconate, malic acid, phenylacetic acid, adipic acid and any combination thereof.

According to a further preferred embodiment of the invention, the Bacillus simplex strain of the invention is positive for: citrate utilization; and/or gelatinase; and/or the reduction of nitrates to nitrites.

According to a further preferred embodiment of the invention, the Bacillus simplex strain of the invention is resistant to lincomycin, preferably present in a concentration ranging from 4-32 μg/ml, and/or susceptible to at least one of the antibiotic agent, preferably present in a concentration ranging from 4-64 μg/ml, selected from: novobiocin, ampicillin, sulbactam, ceflaclor, cefonicid, ceftazidime, ceftriaxone, cefuroxime, ciprofloxacin, levofloxacin, pefloxacin, azithromycin, miokamycin and any combination thereof, and/or intermediately susceptible to at least one of the antibiotic agent selected from: gentamycin, netilmicin, tobramycin, amoxicillin/clavulanic acid, piperacillin, cefixime, ceftazidime, clarithromycin, erithromycin, roxitromycin, fosfomycin, rifampicin, co-trimoxazole and any combination thereof.

According to a further preferred embodiment of the invention, the Bacillus simplex strain of the invention is characterized by a population doubling of 12-90 minutes.

According to a preferred embodiment of the invention, the Bacillus simplex strain of the invention is characterized by a DNA, preferably the DNA of 16S rRNA gene coding, comprising SEQ ID NO: 1 or any sequence characterized by 80-99.9% of identity with SEQ ID NO: 1.

16S ribosomal RNA sequences have been used extensively in the classification and identification of Bacteria and Archaea. The comparison of almost complete 16S rRNA gene sequences has been widely used to establish taxonomic relationships between prokaryotic strains, with 98.65% similarity currently recognized as the cut-off for delineating species. The comparison of the 16S rRNA gene sequence of an isolate against sequences of type strains of all prokaryotic species provides an accurate and convenient way to routinely classify and identify prokaryotes. Bacterial 16S ribosomal RNA (rRNA) genes contain nine “hypervariable regions” (V1-V9) that demonstrate considerable sequence diversity among different bacteria. Preferably, in order to identify and/or to characterize the Bacillus simplex strain of the invention it is advisable to sequencing the full-length 16S rRNA gene comprising at least one, preferably all hypervariable regions from V1 to V9. Preferably the full length 16S rRNA gene sequence comprises SEQ ID NO: 1 or any sequence characterized by 80-99.9% of identity with SEQ ID NO: 1.

According to a further preferred embodiment of the invention, the Bacillus simplex strain of the invention is characterized by a DNA, preferably the DNA of gapA gene coding, comprising SEQ ID NO: 2 or any sequence characterized by 80-99.9% of identity with SEQ ID NO: 2. Glyceraldehyde-3-phosphate dehydrogenase (gapA) partial gene amplification and/or sequencing is a further taxonomic tool, since it is more variable than 16S rRNA coding gene. gapA gene encodes a protein involved in step 1 of the subpathway that synthesizes pyruvate from D-glyceraldehyde 3-phosphate an is part of the pathway glycolysis, which is itself part of carbohydrate degradation.

According to a further preferred embodiment of the invention, the Bacillus simplex strain of the invention is characterized by a DNA, preferably the DNA of pgk gene coding, comprising SEQ ID NO: 3 or any sequence characterized by 80-99.9% of identity with SEQ ID NO: 3.

PhosphoGlycerate Kinase (gpk) partial gene amplification and sequencing is a further taxonomic tool, since is more variable than 16S rRNA coding gene. gpk gene is coding a protein involved in step 2 of the subpathway that synthesizes pyruvate from D-glyceraldehyde 3-phosphate an is part of the pathway glycolysis, which is itself part of carbohydrate degradation.

According to a further preferred embodiment of the invention, the Bacillus simplex strain of the invention is characterized by a DNA, preferably the DNA of uvrA gene coding, comprising SEQ ID NO: 4 or any sequence characterized by 80-99.9% of identity with SEQ ID NO: 4.

UvrABC system protein A (uvrA) partial gene amplification and sequencing is a further taxonomic tool, since is more variable than 16S rRNA coding gene. UvrA is an ATPase and a DNA-binding protein, repair system catalyzes the recognition and processing of DNA lesions.

According to a further preferred embodiment of the invention, the Bacillus simplex strain of the invention is characterized by a DNA comprising: SEQ ID NO: 1 or any sequence characterized by 80-99.9% of identity with SEQ ID NO: 1; and/or SEQ ID NO: 2 or any sequence characterized by 80-99.9% of identity with SEQ ID NO: 2; and/or SEQ ID NO: 3 or any sequence characterized by 80-99.9% of identity with SEQ ID NO: 3; and/or SEQ ID NO: 4 or any sequence characterized by 80-99.9% of identity with SEQ ID NO: 4.

In order to amplify the taxonomic-specific genes reported above any pair of primers located in the DNA sequence of interest can be used. Preferably, the pair of primers are the one disclosed in the examples.

The invention refers also to a further isolated strain that is a member of the genus Paenibacillus, species polymyxa deposited on Mar. 15 2017 at the DSMZ with the accession number DSM32460 and having the following name (denomination): Paenibacillus polymyxa strain VMC10/96.

The Paenibacillus polymyxa strain of the invention is gram positive and is preferably characterized by rod-shaped cells. The colony—when the bacteria are grown on nutrient agar—shows a white color and is characterized by a size having an average diameter of 2-10 millimeters, preferably 2-4 millimeters.

Preferably, the colony's shape of the Paenibacillus polymyxa strain is pale and/or thin, often further showing ameboid spreading. According to a preferred embodiment of the invention, the Paenibacillus polymyxa strain of the invention is able to grow at a temperature around 40° C.

According to a further preferred embodiment of the invention, the Paenibacillus polymyxa strain of the invention is able to use at least one of the carbon source selected from: glycerol, arabinose, ribose, xylose, galactose, glucose, fructose, mannose, mannitol, methyl-aD-glucopyranoside, amygdalin, arbutin, esculin, salicin, cellobiose, maltose, lactose, melibiose, saccharose, trehalose, inulin, raffinose, amidon, glycogen, gentiobiose, turanose, potassium gluconate and any combination thereof. According to a further preferred embodiment of the invention, the Paenibacillus polymyxa strain of the invention is positive for: beta-galactosidase, and/or the acetoin production, and/or gelatinase and/or the reduction of nitrates to nitrites.

According to a further preferred embodiment of the invention, the Paenibacillus polymyxa strain of the invention is resistant to at least one of the antibiotic agent, preferably present in a concentration ranging from 4-32 μg/ml, selected from: cefonicid, ceftazidime, ceftriaxone, ciprofloxacin, miokamycin, co-trimoxazole, lincomycin and any combination therefore; and/or susceptible to at least one of the antibiotic agent, preferably present in a concentration ranging from 4-200 μg/ml, selected from: netilimicin, tobramycin, amoxicillin, ampicillin, pefloxacin, azithromycin, roxitromycin, fosfomycin, rifampicin, and any combination thereof.

According to a further preferred embodiment of the invention, the Paenibacillus polymyxa strain of the invention is characterized by a population doubling of time 37-50 minutes.

According to a preferred embodiment of the invention, the Paenibacillus polymyxa strain of the invention is characterized by:
a DNA, preferably the DNA of 16S rRNA gene coding, comprising SEQ ID NO: 5 or any sequence characterized by 80-99.9% of identity with SEQ ID NO: 5; and/or
a DNA, preferably the DNA of rpoB gene coding, comprising SEQ ID NO: 6 or any sequence characterized by 80-99.9% of identity with SEQ ID NO: 6; and/or
a DNA, preferably the DNA of nifH gene coding, comprising SEQ ID NO: 7 or any sequence characterized by 80-99.9% of identity with SEQ ID NO: 7.

In order to amplify the taxonomic-specific genes reported above any pair of primers following in the DNA sequence of interest can be used. Preferably, the pair of primers are the one disclosed in the examples.

Beta subunit of the RNA polymerase (rpoB) partial gene amplification and sequencing provide comparable phylogenetic resolution to that of the 16S rRNA gene at all taxonomic levels, except between closely related organisms (species and subspecies levels), for which it provided better molecular clock because it is sufficiently conserved, besides being a mono-copy gene. rpoB is a RNA polymerase subunit beta with function of DNA binding.

The nifH partial gene amplification and sequencing provide comparable phylogenetic resolution to that of the 16S rRNA gene at all taxonomic levels, except between closely related organisms (species and subspecies levels), for which it provided better molecular clock because it is sufficiently conserved, besides being a mono-copy gene. The entire nif cluster of Paenibacillus polymyxa is approximately 10.5 kb and encodes 9 genes, the gene nifH with nifD and nifK, encode a Mo-nitrogenase, the enzyme responsible for fixing-nitrogen.

According to a preferred embodiment of the invention, the Bacillus simplex and/or the Paenibacillus polymyxa strain(s) may be used as a single strain (Bacillus simplex or the Paenibacillus polymyxa) or as a combination (Bacillus simplex and the Paenibacillus polymyxa). The combination may be also defined as microbial consortium meaning a group of different species and/or strains of microorganisms with different metabolic activities.

Indeed, the experimental evidence has shown that when used in combination or as consortium some of the biological activities tested significantly improved likely because microbial growth is facilitated or boosted by the metabolites produced by each strain. One possible explanation of this finding could be the accumulation of organic substance and enzymes produced chiefly by one of the microorganism that possibly affects positively the growth of the other microorganism in the consortium. The combined action of the two participants in the consortium will in turn provide beneficial substances, such as polysaccharides, and increasing glucose availability from cellulose, which may cause further boost of the overall soil microbial activity. All these effects promote plant growth. According to a preferred embodiment of the invention, the Bacillus simplex and/or the Paenibacillus polymyxa strain(s) can be used as living microorganisms and/or dead cells and/or killed cells, preferably killed by heating, and/or as spores.

Moreover, the Bacillus simplex and/or the Paenibacillus polymyxa strain(s) may be used as a fresh or frozen sample. Alternatively, the strain(s) is(are) used dry, lyophilized or in liquid suspension, or encapsulated in the form of spores and/or living cells.

The strain(s) of the invention may be cultivated continuously or discontinuously in any medium useful to grow bacteria, in liquid or solid form. Preferably the strain(s) is(are) cultivated on or in a medium (liquid/solid) that preferably comprises: nutrient agar, meat extract, peptone, sodium chloride and yeast extract. The temperature of the culturing process ranges between 25° C. and 30° C.

Moreover, the pH value of the medium ranges preferably between 5 and 8, more preferably the pH is around 7.

Therefore, a further aspect of the invention refers to a medium (broth) obtained/obtainable by culturing one or both the strain(s) of the invention.

The medium may contain the bacteria (the cells), that is the whole culture medium, or the medium may be a cell free medium (without the cells). The cell free medium may be obtained preferably by centrifuging the whole culture medium by using the common standard procedure useful for this purpose, in order to obtain a cell-free medium or supernatant. Therefore, the strain(s) of the invention may be used as culture medium (broth), preferably whole culture medium (comprising cells), or cell-free medium or supernatant (without cells).

Alternatively, the strain(s) of the invention is(are) used as lysate, as extract, preferably cell free extract, as fraction or as metabolite(s) derived from said bacterium(a).

As used herein, culture medium means a solid, liquid or semi-solid nutritive matrix to support the growth of cells (prokaryotic or eukaryotic cells).

In this context, alternative names for culture medium are preferably the following: proliferating medium, expansion medium, growth medium, nutrient medium.

As used herein, “whole culture medium” refers to a solution and/or a suspension containing bacterial cells and derivatives thereof, such as nutrients, metabolites or cellular debris.

As used herein, “supernatant” refers to the liquid part of a culture broth free from the bacterial cells.

As used herein, “lysate” means the solution comprising the material released from the lysis of the bacterial cells.

As used herein, “extract” refers to a specific part of the culture broth, including or not living cells.

As used herein, “cell free extract” means a solution containing all the microbial metabolites, without the presence of living cells.

As used herein, “fraction” means a specific part of the whole culture medium, for example only the solution, only the cells or the like.

As used herein, “metabolite” means one or more intermediate(s) or final product(s) of the bacterial metabolism.

A further aspect of the invention refers to mutants and/or edited strains derived/obtainable from the Bacillus simplex and/or the Paenibacillus polymyxa strain(s) here disclosed.

As used herein, “mutant” means any microorganism obtained/obtainable by direct mutation, selection, or genetic recombination of the Bacillus simplex and/or the Paenibacillus polymyxa strain(s). Mutant strains may be obtained by using any methods known in the art for this purpose, such as mutant selection, chemical mutagenesis, genetic manipulation, physical mutagenesis (radiation) and biological mutagenesis. As used herein, “edited” means preferably gene-edited strains wherein at least one gene of interest is edited/modified by using the common biological tools useful for this purpose, in particular biological tools based on the use of nucleases such as zinc finger nucleases (ZFNs), transcription activator-like effector nucleases (TALENs), and the clustered regularly-interspaced short palindromic repeat (CRISPR)/CRISPR-associated (Cas).

According to a preferred embodiment of the invention, the Bacillus simplex and/or the Paenibacillus polymyxa strain(s) is(are) used in combination with further microorganisms, preferably selected from: bacteria, preferably PGPR (or rhizobacteria), yeasts, mycorrhizae, fungi, any derivatives as disclosed above and any combination thereof.

The PGPR of interest that may be used in combination with the strain(s) of the invention is preferably selected from: Aeromonas rivuli, Agromyces fucosus, Bacillus spp. Bacillus mycoides, Bacillus licheniformis, Bacillus subtilis, Bacillus megaterium, Bacillus pumilus, Bacillus safensis, Microbacterium sp., Nocardia globerula, Stenotrophomonas spp., Pseudomonas spp, Pseudomonas fluorescens, Pseudomonas fulva, Pseudoxanthomonas dajeonensis, Rhodococcus coprophilus, Sphingopyxis macrogoltabida, Streptomyces spp., Enterobacter spp., Azotobacter spp., Azospiriullum spp., Rhizobium spp., Herbaspirillum spp., Lactobaccillus spp., Lactobacillus acidophilus, Lactobacillus buchneri, Lactobacillus delbrueckii, Lactobacillus johnsonii, Lactobacillus murinus, Lactobacillus paraplantarum, Lactobacillus pentosus, Lactobacillus plantarum, Lactococcus tactis, and combinations thereof.

The yeasts of interest that may be used in combination with the strain(s) of the invention is preferably selected from: Candida spp., Candida tropicalis, Saccharomyces spp., Saccharomyces bayanus, Saccharomyces boulardii, Saccharomyces cerevisiae, Saccharomyces exiguous, Saccharomyces pastorianus, Saccharomyces pombe, and combinations thereof.

The mycorrhizae of interest that may be used in combination with the strain(s) of the invention is preferably selected from: Glomus spp., Rhizophagus spp., Septoglomus spp., Funneliformis spp., and combinations thereof.

The fungi of interest that may be used in combination with the strain(s) of the invention is preferably selected from: Trichoderma spp., Trichoderma atroviride, Trichoderma viride, Trichoderma afroharzianum, Paecilomyces spp., Beauveria bassiana., Metarhizium spp., Lecanicillium lecanii, Penicillium spp., Aspergillus spp., Conythyrium minitans, Pythium spp, and combinations thereof.

According to a preferred embodiment of the invention, the Bacillus simplex and/or the Paenibacillus polymyxa strain(s) is(are) used in combination with a plant biostimulant (PBS).

As used herein, according to the current definition of European Biostimulant Industry Council, “biostimulant” refers to a substance(s) and/or micro-organisms or a composition comprising said substance(s) and/or micro-organisms whose function when applied to plants or to the rhizosphere is to stimulate natural processes to enhance/benefit nutrient uptake, nutrient efficiency, tolerance to abiotic stress, and crop quality.

Preferably, said plant biostimulant comprises an extract of algae and/or an extract of microalgae and/or an extract of plant and/or a humic acid and/or a fulvic acid, and/or animal byproducts.

As used herein, algae refer to a functional group of organisms that carry out oxygenic photosynthesis and are not embryophytes. They include both bacterial (cyanobacteria) and eukaryotic organism. The term encompasses organisms that are photoautotrophic. heterotrophic, or mixotrophic, and are typically found in freshwater and marine systems. The term algae include macroalgae (such as seaweed) and/or microalgae.

Preferably said algae are brown algae, more preferably said algae are selected from: Ascophyllum nodosum, Ecklonia maxima, Laminaria saccharina, Laminaria digitata, Fucus spiralis, Fucus serratus, F. vesiculosus, Macrocystis spp., Pelvetia canaliculata, Himantalia elongata, Undaria pinnatifida, Sargassum spp, and combinations thereof. Ascophyllum nodosum is particularly preferred for the purposes of the present invention. As used herein, microalgae refer to any microscopic algae that are unicellular and simple multi-cellular microorganisms, including both prokaryotic microalgae, preferably, cyanobacteria (Chloroxybacteria), and eukaryotic microalgae, preferably green algae (Chlorophyta), red algae (Rhodophyta), or diatoms (Bacillariophyta).

Preferably said microalgae are selected from: Spirulina, Scenedesmus, Nannochloropsis, Haematococcus, Chlorella, Phaeodactylum, Arthrospyra, Tetraselmis, lsochrysis, Synechocystis, Clamydomonas, Parietochloris, Desmodesmus, Neochloris, Dunaliella, Thalassiosira, Pavlova, Navicula, Chaetocerous, and combinations thereof. As used herein, plant means any one of the vast number of organisms within the biological kingdom Plantae. Conventionally the term plant implies a taxon with characteristics of multicellularity, cell structure with walls containing cellulose, and organisms capable of photosynthesis. Modern classification schemes are driven by somewhat rigid categorizations inherent in DNA and common ancestry.

In general, these species are considered of limited motility and generally manufacture their own food. Preferably, they include a host of familiar organisms including trees, forbs, shrubs, grasses, vines, ferns, mosses and crop plants as vegetables, orchards and row crops. For the purpose of the present invention, the whole plant or part thereof may be used for the extraction, preferably said part is selected from: leaves, roots, stems, fruits, flowers, seeds, seedlings, bark, berries, skins, and combinations thereof. Preferably, the plant is selected from: beet, sugar cane, alfalfa, maize, brassica, halophytes, soya, wheat, yucca, quillaja, hop, coffee, citrus, olive, and combinations thereof.

Preferably, the extraction process from plants or from algae or from microalgae is similar. More preferably, said extraction process comprises the following steps: (i) providing a sample of algae and/or a sample of microalgae and/or a sample of plants; and (ii) contacting said sample(s) with an aqueous solution comprising an extraction agent, in other words an extraction solution having an aqueous base.

As used herein, the extraction agent can be a base and/or an acid and/or an enzyme. These kind of extraction agents can be used in any combination or singly.

For the purpose of the present invention, the base is preferable an inorganic base, preferably selected from: NaOH, KOH, Na₂CO₃, K₂CO₃, NH₃, salts thereof, and any combination thereof.

For the purpose of the present invention, the acid is preferably selected from: H₂SO₄, HNO₃, HCl, H₃PO₄, and various acids of organic nature preferably selected from: acetic acid, citric acid, formic acid, butyric acid and ascorbic acid, gluconic acid, and any combination thereof.

For the purpose of the present invention, the enzyme is preferably selected from: papain, trypsin, amylase, pepsin, bromelain and specific enzymes that degrade organic polymers present in the algae, preferably alginases, and any combination thereof.

The selection of the extracting agent to be used for the process depends upon the kind of algae/microalgae/plant to be extracted and/or the molecules/components to be extracted from them.

Preferably, the temperature of the extraction process ranges between −20 and 120° C., more preferably between 20 and 100° C.

Preferably, the extraction time ranges from a few minutes to several hours, more preferably between 30 minutes and 18 hours.

Preferably, the extraction process is realised at atmospheric pressure or at a pressure up to 10 Bar, more preferably at a pressure ranging from 1 to 8 Bar.

The extraction process may be followed by a further step of separating/removing the non-solubilised and/or non-extracted component when it is desirable using only the extract in the formulation of the biostimulant. The removing/separating step is preferably performed by decantation, filtration or centrifugation.

Alternatively, a suspension comprising both the extracted component and the non-extracted component can be used.

Preferably, the concentration of the extract from algae and/or microalgae in the biostimulant ranges from 1 to 60%, preferably it ranges from 5 to 50%, more preferably from 10 to 20%, still more preferably around 15%.

Preferably, the concentration of the plant extract in the biostimulant ranges from 1 to 60%, preferably it ranges from 5 to 50%, more preferably from 10 to 20%, still more preferably around 15%. Preferably, the humic acid is extracted from leonardite, lignite, sub-products of the digestion of urban bio-waste and biochar. The extraction process is performed in water, in alkali, in acidic medium, or by pyrolysis.

Preferably, the concentration of the humic acid in the biostimulant ranges from 1 to 20%.

Preferably, the fulvic acid is extracted from peat, lignite, leonardite, digestion of urban bio-waste, biochar and vegetable materials. The extraction process is performed indifferently in water, in alkali, in acidic medium, and by pyrolysis.

Preferably, the concentration of the fulvic acid in the biostimulant ranges from 1 to 20%, preferably from 5 to 10%.

Preferably, when the strain(s) of the invention is(are) used in combination with the PBS as disclosed above, said PBS is present in a concentration ranging from 5 to 50%, preferably from 10 to 40%, more preferably from 15 to 25%, preferably around 20%.

The bacteria are used in a concentration ranging from 0.01 to 10%, preferably from 0.05 to 5%, more preferably from 0.1 to 1%.

Alternatively, the bacteria are used in a concentration ranging from 10\hat{ }4 to 10\hat{ }12 UFC/g each, preferably from 10\hat{ }6 to 10\hat{ }9 UFC/g, more preferably around 10\hat{ }8 UFC/g. The percentage for PBS refers to the sum of percentage of plant biostimulant components in the final composition. In this context, plant biostimulant components is preferably selected from: plant extracts, seaweed extracts, humic acid, fulvic acid, animal byproducts and combinations thereof.

The percentage of the strain(s) refers to the sum of dried biomasses for each microorganism in 100 g of final composition.

Preferably when the strains are used in combination they are used at the same concentration that preferably is around 0.05% or ranging from 10\hat{ }4 to 10\hat{ }12 UFC/g each, preferably from 10\hat{ }6 to 10\hat{ }9 UFC/g, more preferably around 10\hat{ }8 UFC/g. According to a preferred embodiment of the invention, the Bacillus simplex and/or the Paenibacillus polymyxa strain(s) is(are) used in combination with at least one of the following ingredients:
A nitrogen source, preferably selected from: ammonium phosphates, ammonium nitrate, ammonium sulfate, ammonium thiosulfate, potassium thiosulfate, ammonia, urea, nitric acid, potassium nitrate, magnesium nitrate, calcium nitrate, sodium nitrate, protein hydrolisates of vegetal and animal origin, aminoacids, proteins, yeast lysate, manganese nitrate, zinc nitrate, slow release urea, preferably ureaformaldehyde, similar compounds and combinations thereof; and/or
A phosphorus source, preferably selected from: ammonium phosphates, potassium phosphates, phosphoric acid, sodium phosphates, calcium phosphate, magnesium phosphate, rock phosphate preferably hydroxyapatite and fluoroapatite, phosphorus acid, sodium phosphite, potassium phosphite, calcium phosphite, magnesium phosphite, organic phosphorus compounds, preferably inositol-phosphate, sodium glycerophosphate, ATP, similar compound and combinations thereof; and/or
A potassium source, preferably selected from: potassium acetate, potassium citrate, potassium sulfate, potassium thiosulfate potassium phosphate, potassium phosphite, potassium carbonate, potassium chloride, potassium hydroxide, potassium nitrate, mixed salts of magnesium and potassium, potassium sorbate, potassium ascorbate, organic forms of potassium, and combinations thereof; and/or
A magnesium and/or calcium source, preferably selected from: magnesium nitrate, magnesium sulfate, magnesium chloride, magnesium phosphate, magnesium phosphite, magnesium thiosulfate, magnesium hydroxide, magnesium oxide, mixed salts of potassium and magnesium, mixed salts of magnesium and calcium (dolomite), magnesium acetate, magnesium citrate, magnesium sorbate, and organic forms of magnesium, magnesium carbonate, magnesium formiate, magnesium ascorbate, and combinations thereof; and/or
A sulfur source, preferably selected from: sulfuric acid, sulfates, thiosulfate, sulfated aminoacids, and combinations thereof; and/or
Iron and/or manganese and/or zinc and/or copper source, preferably selected from: iron sulfate, iron oxide, iron hydroxide, iron chloride, iron carbonate, iron phosphate, iron nitrate, chelated iron with EDTA, DTPA, HEDTA, EDDHA, EDDHSA, EDDHCA, EDDHMA, HBED, EDDS; complexed iron with aminoacids, ligninsufonates, humic acid, fulvic acid, gluconic acid, heptagluconic acid, iron citrate, iron malate, iron tartrate, iron acetate, iron lactate, iron ascorbate, organic form of iron, and combinations thereof; and/or
A plant biostimulant (PBS) as disclosed in detail above.

According to a preferred embodiment of the invention, the Bacillus simplex and/or the Paenibacillus polymyxa strain(s) is(are) used in combination with further molecules, preferably proteins, protein hydrolisates, peptides, oligopeptides, peptidoglycans, low-molecular weight peptides, synthetic and natural occurring aminoacids; molasses, polysaccharides, lypopolysaccharides, monosaccharides, disaccharides, oligosaccharides, sulfated oligosaccharides, exopolysaccharides, chitosan. Other molecules that can be advantageously used in combination with the Bacillus simplex and/or the Paenibacillus polymyxa strain(s) are selected from: stress protecting molecules, such as betaines, mannitol, and other polyols with similar effects, and hormones and hormone-like compounds, such as melatonin, auxins, auxin-like compounds, cytokinins, cytokinin-like compounds, gibberellins, gibberellin-like compounds, jasmonates, hormones precursors like polyamines spermine, spermidine, putrescine, and metabolism stimulating substances like vitamins. Other molecules that can be advantageously used in combinations with the Bacillus simplex and/or the Paenibacillus polymyxa strain(s) are selected from: nucleic acids, uronic acids and polymers thereof, glucuronic acids and polymers thereof, small organic acids, such as oxalic and succinic acids. Preferably, said small molecules may be a synthetic and/or naturally derived nucleic acid molecules containing multiple nucleotides, preferably being defined an oligonucleotide when the molecule is 18-25 nucleotides in length and polynucleotides when the molecule is 26 or more nucleotides. Preferably said oligonucleotides or polynucleotides or a mixture of both, include RNA or DNA or RNA/DNA hybrids or chemically modified oligonucleotides or polynucleotides or a mixture thereof.

A further aspect of the invention refers to a composition, preferably an agricultural composition, more preferably a plant biostimulant composition, comprising the Bacillus simplex and/or the Paenibacillus polymyxa strain of the invention as disclosed above, eventually in combination with the further components/ingredients disclosed above and a carrier, preferably an agricultural compatible carrier. Preferably, said further components/ingredients is the PBS and/or the further molecules as previously disclosed.

As used herein “agricultural compatible carrier” refers to any synthetic or natural derived molecule able to deliver the product in an active form in the site of action, preferably said carrier is selected from: surfactants, thickeners, suspension agents, wetting agents, and combinations thereof.

As used herein, surfactant means any molecule able to modify the surface tension of the water, and allowing the product to impact a wider area of the leave and/or root and/or fruit, or any other part of the plant. Preferably said surfactant is selected from: ionic, non-ionic, cationic surfactants, synthetic or naturally derived, preferably alkyl sulfonates, alkylarylsulfonates, ethoxylated alcohols, alkoxylated ethers, ethoxylated esters, alkylpolyglucosides, block copolymers, lignosulfonates, saponins, and the like. As used herein, thickener means any molecule able to modify the rheology of any given composition in the sense of improving the viscosity and stabilize it. Preferably, said thickener is selected from: natural and synthetic gums, lignosulfonates, molasses and the like.

As used herein, suspension agent means any molecule able to surround insoluble particles avoiding settlement and allowing the creation of a stable suspension of insoluble. Preferably, said suspension agent is selected from: natural and synthetic colloids, clays, and their derivatives and the like.

As used herein, wetting agent means any molecule able to avoid fast water evaporation on a given surface and retain moisture for a long time. Preferably, said wetting agent is selected from: glycols, glycerin and their derivatives and the like.

The Bacillus simplex and/or the Paenibacillus polymyxa strain(s) of the invention, eventually, in combination with the further components/ingredients disclosed above and/or the composition disclosed above is/are formulated as: solution, suspension, water-soluble concentrates, dispersable concentrates, emulsifiable concentrates, emulsions, suspensions, microemulsion, gel, microcapsules, granules, ultralow volume liquid, wetting powder, dustable powder, or seed coating formulations.

As shown in the experimental part below, the Bacillus simplex and/or the Paenibacillus polymyxa strain(s) of the invention is(are) able to solubilize macronutrients and/or micronutrients as defined above. Preferably, the strain(s) is(are) able to solubilize macronutrients and/or micronutrients present in the soil and therefore the strain(s) allow(s) said solubilized macronutrients and/or micronutrients being more available for plants that consequently show an improved capability of micro and/or macronutrient's uptake. In other words, thanks to the macronutrients and/or micronutrients solubilizing (mobilizing/dissolving) activity of the strain(s) of the invention, plants improve the up-take capability of said solubilized macronutrients and/or micronutrients, preferably from the soil, and show a better growth.

As define above, said macronutrients can be phosphate, more preferably any inorganic source of phosphate. The Bacillus simplex and/or the Paenibacillus polymyxa strain(s) of the invention, eventually, in combination with the further components/ingredients disclosed above and/or the composition disclosed above can be used to solubilize (mobilize/dissolve) or to improve the dissolution, preferably in the soil, of phosphate, preferably any inorganic source of phosphate, more preferably selected from: phosphate tricalcium phosphate (Ca₃(PO₄)₂), ferric phosphate (FePO₄), and aluminum phosphate (AIPO₄).

In other words, insoluble forms of phosphorus, preferably contained into soils, are converted, through several mechanisms, preferably by organic acids production, chelation, ion-exchange reaction or polymeric substances formation, into soluble forms preferably H₂PO₄⁻ and/or HPO₄²⁻ that are easier to be taken up plants.

According to a further embodiment of the invention, the Bacillus simplex and/or the Paenibacillus polymyxa strain(s) of the invention, eventually, in combination with the further components/ingredients disclosed above and/or the composition disclosed above is(are) able to solubilize (mobilize/dissolve) zinc, preferably any inorganic source of zinc, more preferably selected from: Zinc Oxide (ZnO), Zinc(II) Carbonate (ZnCO₃), and Zinc(II) Phosphate (Zn₃(PO₄)₂).

Preferably, the Bacillus simplex and/or the Paenibacillus polymyxa strain(s) of the invention, eventually, in combination with the further components/ingredients disclosed above and/or the composition disclosed above is(are) able to convert into soluble forms, existing as free Zn²⁺ ions and Zn chelates, by the bacterial production of organic acids such as 5-ketogluconic acid and 2-ketogluconic acid. Indeed, gluconic acids and ketogluconates are sugar acids having multiple conformations, which chelate the metal cations coming from solubilization process.

According to a further embodiment of the invention, the Bacillus simplex and/or the Paenibacillus polymyxa strain(s) of the invention, eventually, in combination with the further components/ingredients disclosed above and/or the composition disclosed above is(are) able to produce siderophores. Preferably, the Bacillus simplex and/or the Paenibacillus polymyxa strain(s) of the invention, eventually, in combination with the further components/ingredients disclosed above and/or the composition disclosed above is(are) able to chelate iron ions (Fe³⁺), making iron more available for plants. As used herein, “siderophores” mean small, high-affinity iron-chelating compounds generally secreted by microorganisms such as bacteria, fungi and grasses.

Siderophores are amongst the strongest soluble Fe3+ binding agents known. These compounds are small proteic molecules generally <1000 Da, although some siderophores are bigger. They are rapidly assembled through short, well-defined metabolic pathways. These molecules comprise lateral chains and functional groups that confer a strong affinity (usually with K_{d}>10³⁰ M⁻¹) to coordinate with the ferric ion (Fe³⁺). Typically, microbial siderophores belong to at least one of class of molecules preferably selected from: catecholates, hydroxamates, and α-carboxylates, depending on the chemical nature of their coordination sites with iron. Preferably, the strain(s) of the invention is able to produce hydroxamates type siderophores. These siderophores form iron chelates by the binding site that is mounted on an L-ornithine derivative. Through this mechanism iron ion (Fe³⁺) is taken from insoluble forms and became available for plants. Iron privation in plants causes as main effect the reduction in photosynthetic activity and as secondary effect the reduction in fruit quality in terms of color, size, sugar content, fruit hardness and taste. Moreover, siderophores support plant growth also by inhibition of soil-borne plant pathogens. Indeed, the siderophores—produced in iron-limited conditions—sequester the less-available iron from the environment and inhibit pathogens by depriving iron.

Therefore, according to a further embodiment of the invention, the Bacillus simplex and/or the Paenibacillus polymyxa strain(s) of the invention, eventually, in combination with the further components/ingredients disclosed above and/or the composition disclosed above can be used to improve the capability of plants to uptake iron preferably from soil.

Moreover, as demonstrated in the experimental data, the strain(s) expresses endoglucanase and therefore the strain(s) of the invention is(are) able to transform/degrade organic matter into soil, contributing to humification and/or improving soil fertility. In this regard, indeed, humus affects soil properties by increasing soil aggregation and its ability to attract and retain nutrients.

Therefore, according to a further embodiment of the invention, the Bacillus simplex and/or the Paenibacillus polymyxa strain(s) of the invention, eventually, in combination with the further components/ingredients disclosed above and/or the composition disclosed above can be used to improve the capability of plants to use complex sugar sources, preferably cellulose and/or derivative thereof.

As already disclosed above, the Bacillus simplex and/or the Paenibacillus polymyxa strain(s) of the invention may be used alone or in combination as a consortium. When used in combination the Bacillus simplex and the Paenibacillus polymyxa strains of the invention are used in the same amount (1:1). Alternatively, the range between the amount of the Bacillus simplex and the Paenibacillus polymyxa strains of the invention varies between 1:10 and 1:100.

Indeed, when tested together, especially in presence of a plant biostimulant, the biological properties discussed above are particularly enhanced as well as the crop yield. In this regard, the synergism between the strains in the presence of the PBS may be due to their ability to degrade the PSB's components and to generate metabolites able to support their growth/activities.

According to a preferred embodiment, the Bacillus simplex and/or the Paenibacillus polymyxa strain(s) of the invention when used as consortium are able to emphasize, preferably to synergize, plants responses to any biostimulant substance. Preferably, a significant yield improvement has been observed when the Bacillus simplex and/or the Paenibacillus polymyxa strain(s) of the invention is(are) used with a biostimulant as defined above.

Therefore, the Bacillus simplex and/or the Paenibacillus polymyxa strain(s) of the invention, preferably in combination with one or more of the further components/ingredients disclosed above and/or the composition as disclose above is(are) particularly useful in agriculture, preferably to improve, preferably into soil, 1) macronutrients and/or micronutrients solubilization (mobilization/dissolution), preferably phosphate and/or zinc solubilization, and/or 2) siderophores production, preferably iron availability, and/or 3) transformation/degradation of organic matter into soil and/or 4) humidity and/or fertility. Consequently, the Bacillus simplex and/or the Paenibacillus polymyxa strain(s) of the invention, preferably in combination with one or more of the further components/ingredients disclosed above and/or the composition as disclose above is(are) particularly useful to improve macronutrients and/or micronutrients plant uptake, phosphate and/or zinc and/or iron uptake from plants. In view of these effects, the Bacillus simplex and/or the Paenibacillus polymyxa strain(s) of the invention, preferably in combination with one or more of the further components/ingredients disclosed above and/or the composition as disclose above is(are) useful to improve plant growth. Preferably, said further components/ingredients is the PBS as previously disclosed.

As used herein, plant means any one of the vast number of organisms within the biological kingdom Plantae. Conventionally the term plant implies a taxon with characteristics of multicellularity, cell structure with walls containing cellulose, and organisms capable of photosynthesis. Modern classification schemes are driven by somewhat rigid categorizations inherent in DNA and common ancestry.

In general, these species are considered of limited motility and generally manufacture their own food. Preferably, they include a host of familiar organisms including trees, forbs, shrubs, grasses, vines, ferns, mosses and crop plants as vegetables, orchards and row crops.

Preferably the plant to be treated is a vegetable plant, more preferably any living organism belonging to the Kingdom Plantae, including both monocotyledonous plants, also called monocots, and dicotyledonous plants, also called dicots. Preferably, the plant can be selected from: Solanaceae, Cucurbitaceae, Graminaceae (Poaceae), Pomaceae, Chenopodiaceae, Brassicaeae, Compositae, Liliaceae, Leguminosae, Rosaceae, Vitaceae, Rutaceae, Oleaceae, Moraceae, Malvaceae, Musaceae, Lauraceae, Anacardiaceae, Juglandaceae, Zingiberaceae, Labiateae, Piperaceae, Cannabaceae, Arecaceae, Punicaceae, Bromeliaceae, Rubiaceae, Theaceae, Caricaceae, Passifloraceae, Asteraceae, Actinidiaceae, Fagaceae, Fabaceae, Ginkoaceae, Simondsiaceae and combinations thereof. More preferably said plant is selected from: tomato, melon, eggplant, pepper, cucumber, zucchini, potato, cauliflower, onion, lettuce, spinach, cabbage, savaoy cabbage, corn, wheat, barley, soybean, peach, apricot, plum, apple, pear, strawberry, grapes, cotton, almonds, various ornamentals and combinations thereof.

All the sequences disclosed in the present invention are listed in the following Table and are further submitted as Sequence Listing. Any sequence having at least 80-99% of identity with the sequences herewith should be considered part of the present disclosure.

TABLE I

| SEQUENCE | SEQ ID NO | NAME |
|---|---|---|
| GACGAACGCTGGCGGCGTGCCTAATACATGCAAGTCGAGCGAATCGATG | SEQ ID NO: 1 | 16S rRNA of the Bacillus simplex |
| GGAGCTTGCTCCCTGAGATTAGCGGCGGACGGGTGAGTAACACGTGGG | | strain VMC 10/70 |
| CAACCTGCCTATAAGACTGGGATAACTTCGGGAAACCGGAGCTAATACCG | | |
| GATACGTTCTTTTCTCGCATGAGAGAAGATGGAAAGACGGTTTACGCTGT | | |
| CACTTATAGATGGGCCCGCGGCGCATTAGCTAGTTGGTGAGGTAATGGC | | |
| TCACCAAGGCGACGATGCGTAGCCGACCTGAGAGGGTGATCGGCCACAC | | |
| TGGGACTGAGACACGGCCCAGACTCCTACGGGAGGCAGCAGTAGGGAA | | |
| TCTTCCGCAATGGACGAAAGTCTGACGGAGCAACGCCGCGTGAACGAAG | | |
| AAGGCCTTCGGGTCGTAAAGTTCTGTTGTTAGGGAAGAACAAGTACCAGA | | |
| GTAACTGCTGGTACCTTGACGGTACCTAACCAGAAAGCCACGGCTAACTA | | |
| CGTGCCAGCAGCCGCGGTAATACGTAGGTGGCAAGCGTTGTCCGGAATT | | |
| ATTGGGCGTAAAGCGCGCGCAGGTGGTTCCTTAAGTCTGATGTGAAAGC | | |
| CCACGGCTCAACCGTGGAGGGTCATTGGAAACTGGGGAACTTGAGTGCA | | |
| GAAGAGGAAAGTGGAATTCCAAGTGTAGCGGTGAAATGCGTAGAGATTTG | | |
| GAGGAACACCAGTGGCGAAGGCGACTTTCTGGTCTGTAACTGACACTGA | | |
| GGCGCGAAAGCGTGGGGAGCAAACAGGATTAGATACCCTGGTAGTCCAC | | |
| GCCGTAAACGATGAGTGCTAAGTGTTAGAGGGTTTCCGCCCTTTAGTGCT | | |
| GCAGCTAACGCATTAAGCACTCCGCCTGGGGAGTACGGCCGCAAGGCTG | | |
| AAACTCAAAGGAATTGACGGGGGCCCGCACAAGCGGTGGAGCATGTGGT | | |
| TTAATTCGAAGCAACGCGAAGAACCTTACCAGGTCTTGACATCCTCTGAC | | |
| AACCCTAGAGATAGGGCTTTCCCCTTCGGGGGACAGAGTGACAGGTGGT | | |
| GCATGGTTGTCGTCAGCTCGTGTCGTGAGATGTTGGGTTAAGTCCCGCAA | | |
| CGAGCGCAACCCTTGATCTTAGTTGCCAGCATTCAGTTGGGCACTCTAAG | | |
| GTGACTGCCGGTGACAAACCGGAGGAAGGTGGGGATGACGTCAAATCAT | | |
| CATGCCCCTTATGACCTGGGCTACACACGTGCTACAATGGATGGTACAAA | | |
| GGGCTGCAAACCTGCGAAGGTAAGCGAATCCCATAAAGCCATTCTCAGTT | | |
| CGGATTGCAGGCTGCAACTCGCCTGCATGAAGCCGGAATCGCTAGTAAT | | |
| CGCGGATCAGCATGCCGCGGTGAATACGTTCCCGGGCCTTGTACACACC | | |
| GCCCGTCACACCACGAGAGTTTGTAACACCCGAAGTCGGTGAGGTAACC | | |
| TTCATGGAGCCAGCCGCCTAAGGTGGGACAGATGATTGGGGTGAAGTCG | | |
| TAACAAGGTAGCCGTATCGGAAGG | | |
| TATATGTTAGCGCACCTTCTTCAATACGATACAATTCATGGATCACTTAAT | SEQ ID NO: 2 | → gapA gene from the Bacillus |
| GAAAAAGTAACAGTTGATGGGGATTACCTTGTTGTTGATGGTCATAAAGTC | | simplex strain VMC 10/70 |
| AAAGTATTGGCTGAACGTGACCCTGCACAATTAGCATGGGGTGAACTAGG | | |
| AGTAGAAGTAGTAGTAGAATCTACAGGACGTTTCACGAAACGTGCAGATG | | |
| CAGCTAAGCATTTAGAAGCTGGCGCGAAAAAAGTAATCATCTCTGCTCCT | | |
| GCATCTGATGAAGATATCACAATCGTCATGGGTGTAAACGAAGATAAATAT | | |
| GATGCAGCTAACCACCATGTAATCTCTAATGCTTCTTGTACAACGAACTGC | | |
| TTAGCTCCATTTGCTAAAGTGCTTCACGAACAATTCGGAATCAAACGCGG | | |
| TATGATGACTACTGTTCACTCTTATACAAATGATCAGCAAATCCTTGATTTG | | |
| CCACATAAAGATTACCGCCGTGCACGTGCAGCTGCCGAGAATATCATTCC | | |
| TACAACAACTGGGGCAGCAAAAGCTGTTGCACTTGTCCTTCCTGAACTTA | | |
| AAGGGAAATTGAATGGTATGGCAATGCGCGTACCTACTCCAAACGTGTCT | | |
| GTTGTCGACCTTGTTGCAGAACTTGAAAAAGACACAACAGTTGAAGAAGT | | |
| TAATGCAGCATTCAAAAAGGCTTCTGAAGGTGAATTAAAAGGAATCCTTGA | | |
| GTACAGCGAACTTCCGCTAGTATCAACTGACTATAACGGTAACCCATCAT | | |
| CT | | |
| CTGCTTCAACTGAAGGAATAGCGCACCATCTCCCAGCCGTTGCCGGCTT | SEQ ID NO: 3 | pgk gene from the Bacillus |
| GCTGATGGAAAAAGAGCTTTCAGTACTTGGAAAAGCCCTATCCAACCCAG | | simplex strain VMC 10/70 |
| AACGTCCTTTTACAGCTATAATTGGCGGAGCAAAAGTAAAGGATAAGATA | | |
| GGCGTTATCGAAAACCTTTTGGAAAAAGTCGATCACTTGATCATTGGTGG | | |
| TGGATTGGGTTATACATTCATTAAAGCGCAAGGTCATGAAATCGGTAATTC | | |
| TTTATTGGAGGAAGACAAAATAGAATTGGCCAAATCTTTCATCGAAAGTGC | | |
| AAAAGAAAAAGGCGTAAAACTTCATTTGCCTATCGATGCAGTCGTAACTG | | |
| CTGAATTTTCACCTGATGCAGAGACGGATAATGTCGATATTGATGCTATTC | | |
| CAAAGGATAAAATGGCTCTTGATATCGGACCAAAAACAAGCGAATTATTTG | | |
| CGGATGTA | | |
| CATTTCCATAGACCAGAAAACGACCAGTCGAAATCCTCGTTCAACCGTAG | SEQ ID NO: 4 | uvrA gene from the Bacillus |
| GGACTGTTACGGAGATCTATGATTATTTAAGGCTGTTATTTGCACGGGTC | | simplex strain VMC 10/70 |
| GGCAGGCCGACCTGTCCCATCCATAATATAGAAATCACCTCACAAACGAT | | |
| AGAGCAAATGGTGGACCGCATTCTTGATTACCCTGAGCGAACCAAGCTTC | | |
| AAGTATTGGCACCGCTAGTCTCAGGCAGAAAAGGGACACATGCGAAAGTT | | |
| TTGGAGGAAGTTAAGAAACAAGGATATGTCCGCATTCGTGTGAACGGGGA | | |
| AATGCATGACCTCAGCGATGAGATCACTCTCGAAAAAAATAAAAAACATTC | | |
| GATTGAAGTCATCATAGACCGGATCGTCATAAAAGAGGGTATCATGGCCA | | |
| GGCTTGCAGATTCATTGGAAAGTGCTTTGCAGCTTGGCGAAGGTAAAGTC | | |
| ATCATTGACGTCATGGGTGAGGAAGAGCTGCTGTTCAGTGAACATCATGC | | |
| TTGTCCATACTGCGGATTTTCGATTGAAGAGTTAGAGCCTAGA | | |
| ATGTTTTCCTTCAATAGCCCGTTTGGGGCCTGCCCGGATTGTGATGGCTT | | |
| GGGGGCAAGGCTTGAGGTGGACCGTGATCTGGTCATTCCGAATAACGAA | | |
| TTAAGCCTCCGTCAACATGCAATTGCGCCATGGGAGCCGACTAGTTCTCA | | |
| ATATTACCCTCAGCTCCTTGAAGCGGTGGCAAACCATTATGGGATAGATA | | |
| TGGATGTGTCCGTTAAGGATTTACCGGAAGAGAAGATGGATAAGGTCTTG | | |
| CTTGGTTCAGGCAAAGATAAGATATATTTCCGTTATAAAAATGATTTTGGA | | |
| AGAGTGCAAGAAGGATATATTCCTTTTGAAGGAGTTTTAAGAAACATCGAA | | |
| AGGCGCTTCAAGGAGACGAGTTCTGACTTTATTCGTGAGCAAATGCAGAA | | |
| ATACATGTCAGAACATCACTGTCCAACCTGTAAGGGTCATCGATTGAAAAA | | |
| AGAGAGTCTTTCCGTGCTCATTCAAGGGGTCCATATTAGTGAAACGACAG | | |
| CTTTATCAGTGGAGGATGCGTTAGTATTTTTCGATGAGCTTGATTTGACTG | | |
| AAAAAGAAGCTGCAATTGCGAGATTGATTTTACGTGAAATTCGTGAGCGG | | |
| CTCGGTTTCCTGGCTAATGTAGGTTTGGAATATTTGACGTTGAGCAGGGC | | |
| AGCGGGAACTTTATCAGGCGGTGAAGCGCAGCGTATAAGATGGCGACGC | | |
| AGATCGGTTCCCGATTGACTGGAGTC | | |
| GACGAACGCTGGCGGCGTGCCTAATACATGCAAGTCGAGCGGGGTTGTG | SEQ ID NO: 5 | 16S rRNA of the Paenibacillus |
| TAGAAGCTTGCTTCTACATAACCTAGCGGCGGACGGGTGAGTAACACGTA | | polymyxa strain VMC 10/96 |
| GGCAACCTGCCCACAAGACAGGGATAACTACCGGAAACGGTAGCTAATA | | |
| CCCGATACATCCTTTTCCTGCATGGGAGAAGGAGGAAAGACGGAGCAAT | | |
| CTGTCACTTGTGGATGGGCCTGCGGCGCATTAGCTAGTTGGTGGGGTAA | | |
| AGGCCTACCAAGGCGACGATGCGTAGCCGACCTGAGAGGGTGATCGGC | | |
| CACACTGGGACTGAGACACGGCCCAGACTCCTACGGGAGGCAGCAGTA | | |
| GGGAATCTTCCGCAATGGGCGAAAGCCTGACGGAGCAACGCCGCGTGA | | |
| GTGATGAAGGTTTTCGGATCGTAAAGCTCTGTTGCCAGGGAAGAACGTCT | | |
| TGTAGAGTAACTGCTACAAGAGTGACGGTACCTGAGAAGAAAGCCCCGG | | |
| CTAACTACGTGCCAGCAGCCGCGGTAATACGTAGGGGGCAAGCGTTGTC | | |
| CGGAATTATTGGGCGTAAAGCGCGCGCAGGCGGCTCTTTAAGTCTGGTG | | |
| TTTAATCCCCGAGGGCTCAACTTTCGGGTCGCACTGGAAACTGGGGAGC | | |
| TTGAGTGCAGAAGAGGAGAGTGGAATTCCACGTGTAGCGGTGAAATGCG | | |
| TAGAGATGTGGAGGAACACCAGTGGCGAAGGCGACTCTCTGGGCTGTAA | | |
| CTGACGCTGAGGCGCGAAAGCGTGGGGAGCAAACAGGATTAGATACCCT | | |
| GGTAGTCCACGCCGTAAACGATGAATGCTAGGTGTTAGGGGTTTCGATAC | | |
| CCTTGGTGCCGAAGTTAACACATTAAGCATTCCGCCTGGGGAGTACGGTC | | |
| GCAAGACTGAAACTCAAAGGAATTGACGGGGACCCGCACAAGCAGTGGA | | |
| GTATGTGGTTTAATTCGAAGCAACGCGAAGAACCTTACCAGGTCTTGACA | | |
| TCCCTCTGACCGGTCTAGAGATAGACCTTTCCTTCGGGACAGAGGAGACA | | |
| GGTGGTGCATGGTTGTCGTCAGCTCGTGTCGTGAGATGTTGGGTTAAGT | | |
| CCCGCAACGAGCGCAACCCTTATGCTTAGTTGCCAGCAGGTCAAGCTGG | | |
| GCACTCTAAGCAGACTGCCGGTGACAAACCGGAGGAAGGTGGGGATGAC | | |
| GTCAAATCATCATGCCCCTTATGACCTGGGCTACACACGTACTACAATGG | | |
| CCGGTACAACGGGAAGCGAAATCGCGAGGTGGAGCCAATCCTAGAAAAG | | |
| CCGGTCTCAGTTCGGATTGTAGGCTGCAACTCGCCTACATGAAGTCGGAA | | |
| TTGCTAGTAATCGCGGATCAGCATGCCGCGGTGAATACGTTCCCGGGTC | | |
| TTGTACACACCGCCCGTCACACCACGAGAGTTTACAACACCCGAAGTCG | | |
| GTGGGGTAACCCGCAAGGGAGCCAGCCGCCGAAGGTGGGGTAGATGAT | | |
| TGGGGTGAAGTCGTAACAAGGTAGCCGTATCGGAAGGTGC | | |
| TTGGCAGGACATCTTGTTCAATATGGTCGACGCACTCGGCGCAGTTATGC | SEQ ID NO: 6 | rpoB gene from the Paenibacillus |
| ACGTATTAATGAGGTACTCGAGGTTCCGAACCTGATTGAGATCCAACAAA | | polymyxa strain VMC 10/96 |
| AATCCTATGATTGGTTTTTGGAGGAAGGATTAAGGGAAATGTTTCGGGAT | | |
| ATTTCTCCAATTCAGGATTTCACTGGAAATCTGATTCTGGAGTTTATCGAC | | |
| TATTCTCTCGGAGAACCCAAATATACCGTTGACGACGCAAAGGAACGCGA | | |
| CGTTACGTATGCAGCACCGCTTCGGGTAAAAGTCCGGCTTATTAATAAAG | | |
| AAACCGGGGAAGTGAAAGAGCAGGAAGTATTCATGGGAGACTTCCCGCT | | |
| GATGACTGAAACGGGTACGTTTATTATTAACGGTGCGGAACGGGTTATTG | | |
| TCAGCCAGTTGGTTCGCTCTCCCAGCGTCTATTTCAGCACAAAAGTCGAC | | |
| AAGAATGCGAAAACAACATACACCGCAACGGTAATTCCTAACCGGGGGG | | |
| CTTGGCTCGAACTGGAGATGGATGCGAAGGATATTATCTATGTCCGGATT | | |
| GACCGTACCCGTAAAATTCCGGTTACGGTGTTGCTGCGTGCGCTGGGCT | | |
| TTGGCACTGATGCTGAGATTCTGGATTTGCTCGGCAATGACGAATATATC | | |
| CGCAACACACTTGATAAAGACAACACGGATTCCACCGAGAAAGCGCTGAT | | |
| TGAAATTTATGAGCGTCTTCGTCCAGGTGAGCCGCCTACACTGGATAACG | | |
| CAAAGAGCTTGCTAGTTGCTCGCTTCTTTGATCCTAAACGTTATGATCTGG | | |
| CCAACGTAGGCCGTTACAAAATCAATAAAAAGCTTCACATTAAAAACCGTT | | |
| TGTTCAATCAACGACTTGCTGAGACTTTGATTGATACAACAACTGGTGAAA | | |
| TCATCGCTGAAGCCGGTCAAATGGTAGATCGCCGCCTGTTGGACGAGAT | | |
| TTTGGCCCAACTGGAGGAATCAGTAGGACATCGTACGTATCATGTTGCGA | | |
| GTGGTGTGCTAGAAAGCAATGATATTCCACTTCAAACAATCGATGTGTTCT | | |
| CACCAATTGAGGATGGTAAAGTAGTAAAACTGATTGCTAACGGAAATATTG | | |
| ATAAATCGGTTAAGAATATTACGCCTGCCGATATTATTTCCTCCATCAGTT | | |
| ATTTTATTAACTTGCTTCACGGAATCGGAAGTACGGACGATATTGACCATT | | |
| TGGGTAACCGTCGTTTGCGTTCTGTAGGTGAGTTGCTCCAAAACCAGTTC | | |
| CGTATCGGTTTATCCCGTATGGAACGCGTAGTGCGCGAAAGAATGTCGAT | | |
| TCAGGATGCTAATGTAATTACGCCACAGGCATTGATTAACATACGTCCAGT | | |
| AATTGCTTCGATTAAAGAGTTCTTTGGTAGCTCGCAGCTGTCTCAGTTTAT | | |
| GGATCAGACGAACCCGCTTGCTGAATTAACGCACAAACGTCGTCTGTCCG | | |
| CACTCGGACCCGGCGGTTTGACGCGCGAACGCGCGGGCATGGAAGTGC | | |
| GTGACGTCCATCCGAGTCACTACGGCCGTATGTGTCCTATCGAGACACCA | | |
| GAGGGACCAAACATTGGTTTGATCAACTCTTTGTCCACTTTTGCACGCATT | | |
| AACGAGTATGGATTTATCGAAGCTCCTTATCGTTGGGTAGATCCAAAAAC | | |
| CGGAAAAGTTACAGACCAGATTGATTACCTGACTGCTGATGAAGAAGATA | | |
| ACTACATTGTAGCTCAGGCGAATGCGGAATTGACGGAGGAAAACACCTTT | | |
| AAGGATGAGGTTGTCATTGTCCGTTATAACAAACAGTCTGATAACATTATT | | |
| CCGATGGCTAGTAGCCGTGTCGATTACATGGACGTATCGCCTAAACAGGT | | |
| CGTATCGGTCGCGACTGCACTGATTCCGTTCTTGGAGAATGATGACTCTA | | |
| ACCGCGCATTGATGGGTTCCAACATGCAGCGTCAGGCTGTCCCGCTTCT | | |
| GATTCCGAAGTCTCCATTGGTCGGAACAGGAATGGAGCACAAGTCTGCAA | | |
| AAGATTCCGGTGTTTGCGTTGTATCCAAATACAACGGAGTTATCGAACGTT | | |
| CTTCGGCTAACGAAATTTGGTTGCGTCGTATCGAAACTGTAGATGGCGCT | | |
| GAAGTGAAGGGCGACATTGTTAAGTATAAATTACACAAATTTATGCGATCT | | |
| AACCAAGGAACTTGCATCAACCAACGTCCGATTGTGAACAGAGGAGATAT | | |
| TGTCAAAGTTGGCGATATTCTTGCGGATGGTCCATCTACAGAGATGGGTG | | |
| AGTTGGCGTTGGGACGTAACGTTGTCGTTGCCTTCATGACTTGGGAAGGT | | |
| TACAACTACGAGGATGCGATCTTGCTGAGTGAGAAACTGGTTAAGGAAGA | | |
| TGTATACACCTCGATCCATATTGAGGAATACGAATCCGAGGCTCGTGACA | | |
| CGAAGCTTGGACCGGAAGAAATCACTCGCGACATTCCAAATGTCGGTGAA | | |
| GAAGCGCTTCGCAACTTGGATGAGCGTGGAATCATACGTATTGGTGCTGA | | |
| AATTGGCGCAGGTGACATTCTCGTTGGTAAAGTAACACCTAAAGGTGTGA | | |
| CTGAATTGACAGCTGAAGAACGTCTCTTACACGCAATCTTTGGTGAGAAG | | |
| GCACGCGAGGTTCGCGATACTTCTTTGAGAGTTCCTCACGGAACAGACG | | |
| GGATTGTTGTAGATGTAAAGGTATTTACACGTGAAAATGGCGATGAACTG | | |
| CCACCAGGTGTAAATCAGTTGGTTCGTGTATATATTGCTCAAAAACGTAAA | | |
| ATTTCCGAAGGCGATAAAATGGCTGGACGTCACGGTAACAAGGGTGTCG | | |
| TTGCTCGTATTTTGCCTGAAGAAGATATGCCGTTCCTGCCGGATGGCACA | | |
| CCAGTACAGGTCGTTCTGAACCCGCTGGGCGTACCTTCACGGATGAACA | | |
| TCGGACAGGTGCTTGAAGTCCATCTGGGTATGGCTGCAATGCGTCTTGGT | | |
| ATTCATGTGGCAACTCCAGTATTCGATGGTGCCAAGGAATATGACGTATT | | |
| CGATACAATGGAAGAGGCAGGCATGCAGCGTAATGGTAAGACTGTGTTG | | |
| TATGACGGACGTACGGGTGATCGTTTTGAACGTGAAGTTACTGTCGGTGT | | |
| CATGCACATGATCAAACTGGCGCACATGGTCGATGATAAAATCCATGCTC | | |
| GTTCTACAGGTCCTTACTCTCTCGTTACGCAACAACCATTGGGTGGTAAA | | |
| GCTCAATTCGGTGGACAGCGCTTCGGGGAGATGGAAGTATGGGCATTGG | | |
| AAGCCTACGGTGCAGCGTACACGCTTCAGGAAATTTTGACTGTGAAATCT | | |
| GATGATGTGGTTGGACGTGTTAAAACTTACGAATCCATTGTCAAAGGTGA | | |
| AAATGTACCTGAACCGGGTGTTCCAGAATCATTTAAGGTCTTGATCAAAGA | | |
| GCTGCAAAGCTTGGGTATGGACGTGAAGATTCTGTCTGAAGACGAACAAG | | |
| AGATCGAAATGAGAGAGCTTGATGATGAGGATGACACAACCGGCGATAA | | |
| CT | | |
| GCTACATCTGGCTGCTGAAAGGGGCACGGTAGAGGATTTGGAGCTGGAG | SEQ ID NO: 7 | nifH gene from the Paenibacillus |
| GATGTTGTCCAGAAGGGCTTCGGTGACATTCTGAACGTGGAATGCGGCG | | polymyxa |
| GGCCAGAGCCTGGTGTCGGCTGTGCAGGACGCGGCATCATCACAGCCAT | | |
| TAATTTTCTGGAGGAAGAGGGGGCCTACGAAGGGCTGGATTTTGTTTCCT | | |
| ACGATGTACTGGGGGACGTCGTGTGCGGGGGCTTTGCCATGCCCATCCG | | |
| CGAAAACAAGGCCCAGA | | |
| AGAGTTTGATCCTGGCTCAG | SEQ ID NO: 8 | Forward |
| | | (E8F) |
| AAGGAGGTGATCCANCC | SEQ ID NO: 9 | Reverse |
| | | (E1541R) |
| AACAGATGCTAATATGTTAGC | SEQ ID NO: 10 | Forward |
| | | (gapA-F) |
| GATTTGTAGAAGATGGG | SEQ ID NO: 11 | Reverse |
| | | (gapA-R) |
| CACCGTGCACATGCTTCAAC | SEQ ID NO: 12 | Forward |
| | | (pgk-F) |
| GGACTTTTGATTACATCCGC | SEQ ID NO: 13 | Reverse |
| | | (pgk-R) |
| TTAGGCCAAGTGGATAAACC | SEQ ID NO: 14 | Forward |
| | | (uvrA-F) |
| GCATCGTTATCCCTCTGATG | SEQ ID NO: 15 | Reverse |
| | | (uvrA-R) |
| AACATCGGTTTGATCAAC | SEQ ID NO: 16 | Forward |
| | | (rpo-B-1698f) |
| CGTTGCATGTTGGTACCCAT | SEQ ID NO: 17 | Reverse |
| | | (rpo-B-2041r) |
| GGCTGCGATCCVAAGGCCGAYTCVACCCG | SEQ ID NO: 18 | Forward |
| | | (nifH-F) |
| CTGVGCCTTGTTYTCGCGGATSGGCATGGC | SEQ ID NO: 19 | Reverse |
| | | (nifH-R) |

### Example

The present invention will be described in detail by means of the following examples. The following examples are for illustrative purposes and are not intended to limit the scope of the invention.

Isolation of Novel Bacterial Strains of the Invention

Isolation of the bacterial strains of the invention have been done starting from soil samples coming from Italy.

In more details, 10 grams of each soil sample have been taken and diluted into 90 ml of distilled water.

After homogenization, serial dilution up to 10⁻⁵ have been done. One ml of each serial dilution has been smeared on a nutritive agar plate and incubated for 24 h at 30° C. After incubation period, the most relevant colonies found on the highest dilution (10⁻⁵), have been picked and plated again on nutrient agar plates, incubated for 24 h at 30° C. Through this process we obtained one pure colony from each of the soil samples, later identified as the Bacillus simplex and the Paenibacillus polymyxa of the invention.

The strains have been deposited under Budapest Treaty with the Deutsche Sammlung von Mikroorganismen and Zellkuturen (DSMZ) on Mar. 15 2017 with the following names: Bacillus simplex VMC 10/70 having the deposit number DSM 32459, and Paenibacillus polymyxa VMC 10/96 having the deposit number DSM 32460.

Identification and Characterization of Bacterial Strains

MALDI-TOF Mass Spectrometry Bacterial Identification

Currently microorganisms are best identified using 16S rRNA and 18S rRNA gene sequencing. However, in recent years matrix assisted laser desorption ionization-time of flight mass spectrometry (MALDI-TOF MS) has emerged as a potential tool for microbial identification and diagnosis. During the MALDI-TOF MS process, microbes are identified using either intact cells or cell extracts. In this case, intact cells were tested. Bacillus simplex VMC 10/70 and Paenibacillus polymyxa VMC 10/96 strains of the present invention were characterized using a Microflex™ MALDI-TOF (Matrix-assisted laser desorption ionization—time-of-flight) mass spectrometer (Bruker Daltonics, Leipzig, Germany). Initial manual/visual estimation of the mass spectra was performed using the FlexAnalysis 2.4 software (Bruker Daltonik GmbH, Germany). For automated data analysis, raw spectra were processed using the MALDI BioTyper 1.1 software (Bruker Daltonik GmbH, Germany) with default settings. The smoothing, normalization, baseline subtraction and peak picking was carried out by the software, thereby creating a list of the most significant peaks of a spectrum (m/z values with a given intensity). Samples were prepared according to manufacturers' instructions.

Briefly, after 24 hours of cultivation on Nutrient Broth (NB) at 30° C., a single colony was transferred with a toothpick onto the MALDI steel target plates in triplicate. Spots were overlaid with 1 μl of a saturated solution of -cyano-4-hydroxycinnamic acid (Sigma-Aldrich) in organic solution (50% acetonitrile, 2.5% trifluoroacetic acid), air-dried within minutes at room temperature and directly screened.

Spectra were recorded by Flex Control software (Bruker Daltonics, Bremen, Germany) in a linear positive mode at a laser frequency of 200 Hz in the range from 2 to 20 kDa. In order to assess the reproducibility of MALDI-TOF-MS identification, strains were tested in triplicate (analyses were performed on three different days and starting from different cultures).

For each measurement, at least 300 individual spectra (30 laser shots at 10 different spot positions) were collected and averaged. External calibration was performed with the Bruker bacterial test standard (Bruker Daltonics, Bremen, Germany).

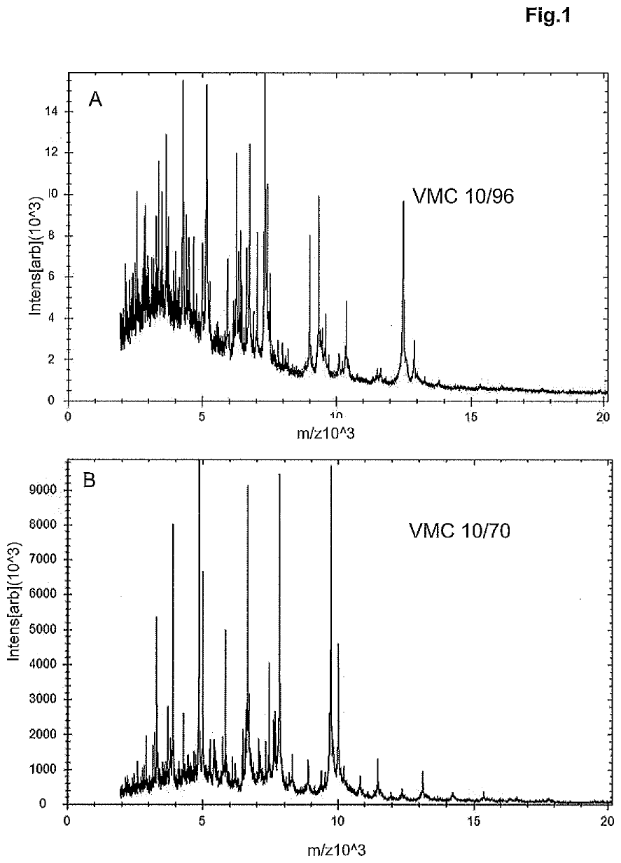

FIG. 1 show MALDI-TOF MS mass spectra of Paenibacillus polymyxa 10/96 (A) and Bacillus simplex strain VMC 10/70 (B), respectively. Each bar in the graphs represents a different protein expressed by the microorganism and the intensity of the bars represents the concentration of the proteins within the microbial cell. Since proteins are a direct expression of genome and genome of each strains is unique, thus the proteomic profile of each strain is unique and can be used as fingerprint. The most and unique representative signal for each one of the strains are in the range from 2 to about 12 KDa using spectrum m/z 10\hat{ }3.

16S-rDNA Sequencing

The 16SrRNA gene sequences of the strains of the invention were determined by direct sequencing of PCR-amplified 16S rDNA.

For the amplification the following primers were used:

| Name | Sequence | SEQ ID NO |
|---|---|---|
| Forward | AGAGTTTGATCCTGGCTCAG | SEQ ID NO: 8 |
| (E8F) | | |
| Reverse | AAGGAGGTGATCCANCCRCA | SEQ ID NO: 9 |
| (E1541R) | | |

The amplification protocol is the following.

| Temperature ° C. | Time | Cycle |
|---|---|---|
| 95 | 5′ | 1X |
| 95; 59; 72 | 30″; 20″; 1.3′ | 30X |
| 72 | 10′ | 1X |

The 16S rDNA sequences of the strains of the invention are set forth in the sequence Listing as indicated in Table I.

The Sanger sequencing was followed by alignment and phylogenetically analysis of the obtained data with the 16S rRNA sequences from the Type Strains.

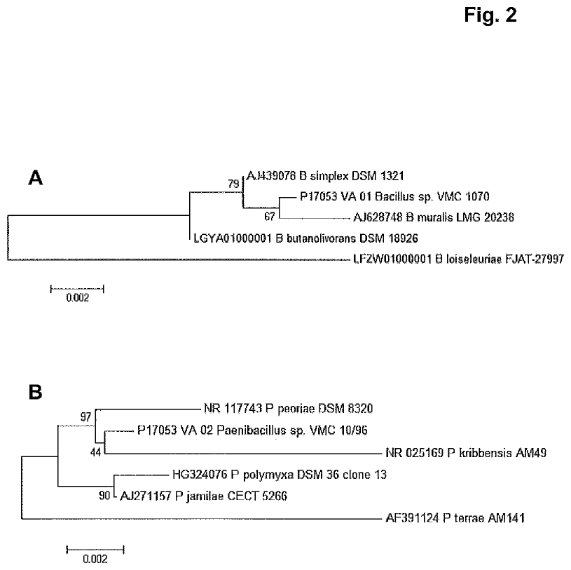

According the analysis of the 16S rRNA sequence:
1) The strain Bacillus simplex VMC 10/70 is related to the species B. simplex and Bacillus muralis as it shares 99.66% and 99.60% of sequence similarity with the 16S rRNA sequence of the corresponding Type Strains (Table II and FIG. 2A).

TABLE II

| Target species | NCBI Acc. | Simi- | |
|---|---|---|---|
| (Type Strain) | No. | larity * | No. Diff.** |
| Bacillus simplex | AJ439078 | 99.66% | 5/1503 |
| DSM 1321 | | | |
| Bacillus muralis | AJ628748 | 99.60% | 6/1503 |
| LMG 20238 | | | |
| Bacillus butanolivorans | LGYA01000001 | 99.60% | 6/1503 |
| DSM 18926 | | | |
| Bacillus loiseleuriae | LFZW01000001 | 97.73% | 34/1503 |
| FJAT-27997 | | | |

* Similarity in terms of percentage, calculated as the ratio between the number of the matching nucleotides and the total number of the nucleotides of the compared sequences. Only the possible degenerated sites due to overlapping peaks in the electropherogram have not been considered in the similarity assessment.
**Number of Single Nucleotide Polymorphisms (SNPs) and insertion/deletion of nucleotides between the query sequence and the most similar sequence in the database.

2) The strain Paenibacillus polymyxa VMC 10/96 is phylogenetically related to the species Paenibacillus peoriae, within the P. polymyxa group, which includes Paenibacillus jamilae, Paenibacillus brasilensis, Paenibacillus kribensis and Paenibacillus terrae, besides P. polymyxa (Table III and FIG. 2B).

TABLE III

| Target species | NCBI Acc. | Simi- | |
|---|---|---|---|
| (Type Strain) | No. | larity * | No. Diff.** |
| Paenibacillus peoriae | NR_117743 | 99.27% | 11/1514 |
| DSM 320 | | | |
| Paenibacillus polymyxa | HG324076 | 99.07% | 14/1514 |
| DSM 36, clone 13 | | | |
| Paenibacillus jamilae | AJ271157 | 98.94% | 16/1514 + |
| CECT 5266 | | | 6N*** |
| Paenibacillus kribbensis | NR_025169 | 98.74% | 19/1514 |
| AM49 | | | |
| Paenibacillus brasilensis | NR_025106 | 98.55% | 20/1384 **** |
| PB172 | | | |
| Paenibacillus terrae | AF391124 | 98.08% | 29/1514 |
| AM141 | | | |

* Similarity in terms of percentage, calculated as the ratio between the number of the matching nucleotides and the total number of the nucleotides of the compared sequences. Only the possible degenerated sites (N) due to overlapping peaks in the electropherogram have not been considered in the similarity assessment;
**The sum of Single Nucleotide Polymorphisms (SNPs) and insertion/deletion of nucleotides differentiating the query sequence and the compared sequence;
***The sequence of P. jamilae shows 6 degenerated nucleotides which have not been considered for the % similarity;
**** The best sequence from the Type Strain of Paenibacillus brasilensis is 1384 bp long.

In order to obtain even finer resolution for the identification and/or characterization, a concatenated set of protein-encoding genes were included (gapA, pgk, uvrA for Bacillus simplex VMC 10/70 and rpoB and nifH for Paenibacillus polymyxa VMC 10/96). Partial amplification and sequencing is a further taxon-specific gene-based approach providing an alternate valuable methodology to carry out the taxonomic classification of newly sequenced or existing bacterial genomes.

gapA Sequencing

The gapA gene sequences of the strains of the invention were determined by direct sequencing of PCR-amplified gapA.

For the amplification the following primers were used:

| Name | Sequence | SEQ ID NO |
|---|---|---|
| Forward | AACAGATGCTAATATGTTAGC | SEQ ID NO: 10 |
| (gapA-F) | | |
| Reverse | GATTTGTAGAAGATGGG | SEQ ID NO: 11 |
| (gapA-R) | | |

The amplification protocol is the following.

| Temperature ° C. | Time | Cycle |
|---|---|---|
| 94 | 5′ | 1X |
| 94; 55; 72 | 1′; 1′; 1.3′ | 35X |
| 72 | 10′ | 1X |

The gapA sequences of the strain of the invention are set forth in the sequence Listing as indicated in Table I.

The Sanger sequencing was followed by alignment, Multilocus sequence typing and phylogenetically analysis of the obtained data with the gapA sequences from the Type Strains.

pgk Sequencing

The pgk gene sequences of the strains of the invention were determined by direct sequencing of PCR-amplified pgk.

For the amplification the followina primers were used:

| Name | Sequence | SEQ ID NO |
|---|---|---|
| Forward | CACCGTGCACATGCTTCAAC | SEQ ID NO: 12 |
| (pgk-F) | | |
| Reverse | GGACTTTTGATTACATCCGC | SEQ ID NO: 13 |
| (pgk-R) | | |

The amplification protocol is the following.

| Temperature ° C. | Time | Cycle |
|---|---|---|
| 94 | 5′ | 1X |
| 94; 63; 72 | 1′; 1′; 1.3′ | 35X |
| 72 | 10′ | 1X |

The pgk sequences of the strains of the invention are set forth in the Sequence Listing as indicated in Table I.

The Sanger sequencing was followed by alignment, multilocus sequence typing and phylogenetically analysis of the obtained data with the pgk sequences from the Type Strains.

uvrA Sequencing

The uvrA gene sequences of the strains of the invention were determined by direct sequencing of PCR-amplified uvrA.

For the amplification the following primers were used:

| Name | Sequence | SEQ ID NO |
|---|---|---|
| Forward | TTAGGCCAAGTGGATAAACC | SEQ ID NO: 14 |
| (uvrA-F) | | |
| Reverse | GCATCGTTATCCCTCTGATG | SEQ ID NO: 15 |
| (uvrA-R) | | |

The amplification protocol is the following.

| Temperature ° C. | Time | Cycle |
|---|---|---|
| 94 | 5′ | 1X |
| 94; 60; 72 | 1′; 1′; 1.3′ | 35X |
| 72 | 10′ | 1X |

The uvrA sequences of the strains of the invention are set forth in the Sequence Listing as indicated in Table I.

The Sanger sequencing was followed by alignment, multilocus sequence typing and phylogenetically analysis of the obtained data with the 16S rRNA sequences from the Type Strains.

rpoB Sequencing

The rpoB gene sequences of the strains of the invention were determined by direct sequencing of PCR-amplified rpoB.

For the amplification the following primers were used:

| Name | Sequence | SEQ ID NO |
|---|---|---|
| Forward | AACATCGGTTTGATCAAC | SEQ ID NO: 16 |
| (rpoB-1698f) | | |
| Reverse | CGTTGCATGTTGGTACCCAT | SEQ ID NO: 17 |
| (rpoB-2041r) | | |

The amplification protocol is the following.

| Temperature ° C. | Time | Cycle |
|---|---|---|
| 94 | 5′ | 1X |
| 94; 60; 72 | 1′; 1′; 1.3′ | 35X |
| 72 | 10′ | 1X |

The rpoB sequences of the strains of the invention are set forth in the Sequence Listing as indicated in Table I.

The Sanger sequencing was followed by alignment, multilocus sequencing typing and phylogenetically analysis of the obtained data with the rpoB sequences from the Type Strains.

nifH Sequencing

The nifH gene sequences of the strains of the invention were determined by direct sequencing of PCR-amplified nifH.

For the amplification the following primers were used:

| Name | Sequence | SEQ ID NO |
|---|---|---|
| Forward | GGCTGCGATCCVAAGGCCGAYTC | SEQ ID NO: 18 |
| (nifH-F) | VACCCG | |
| Reverse | CTGVGCCTTGTTYTCGCGGATSG | SEQ ID NO: 19 |
| (nifH-R) | GCATGGC | |

The amplification protocol is the following.

| Temperature ° C. | Time | Cycle |
|---|---|---|
| 72 | 10′ | 1X |
| 94 | 5′ | 1X |
| 94; 63; 72 | 1′; 1′; 1.3′ | 35X |

The nifH sequences of the strains of the invention are set forth in the Sequence Listing as indicated in Table I.

The Sanger sequencing was followed by alignment and phylogenetically analysis of the obtained data with the nifH sequences from the Type Strains.

The results of the phylogenetic analysis of the gapA, pgk, uvrA, rpoB and nifH, both singly and in concatenated sequence, assessed that the strain B. simplex VMC 10/70 belongs to the specie B. simplex. Moreover, the strain B. simplex VMC 10/70 is clearly different from any other strain whose whole genome is available, or, at least the gapA, uvrA and pgk gene sequences are available. Depending on the specific phylogenetical marker gene, a group of 12 to 13 B. simplex strains was considered here, i.e. all the strains which genetic sequences are available in public databases. The strain B. simplex V MC 10/70 can be therefore considered a novel B. simplex strain.

The results of the phylogenetic analysis carried out with partial rpoB and nifH gene sequences, both singly and in concatenated sequence, assessed that the strain Paenibacillus polymyxa VMC 10/96 belongs to group of species represented by Paenibacillus polymyxa. Moreover, the strain P. polymyxa VMC 10/96 is clearly different from any other strain whose whole genome is available, or, at least the rpoB and nifH gene sequences are available. Depending on the specific phylogenetical marker gene, a group P. polymyxa strains was considered here, i.e. all the strains which genetic sequences are available in public databases. The strain P. polymyxa VMC 10/96 can be therefore considered a novel P. polymyxa strain.

Morphological and Physiological Characterization

The color and the colony morphology of the strains have been characterized by direct observation of the microbial strains after growing on Nutrient Agar (NA) plates. In particular, the bacterial strains have been cultured on NA plates for 24 h at 30° C. After incubation period, bacterial colonies appear on the plates and color and morphology can be observed.

The motility and the colony size have been characterized by using optical microscope with 100× magnitude. In particular, the bacterial strains have been cultured on NA plates for 24 h at 30° C. After incubation period, one bacterial colony has been picked and smeared into 1 ml water drop on a glass slide. The glass slide is then observed under optical microscope with 100× magnitude.

Gram staining is determined by using the Gram staining kit.

The growth of the bacterial strains at different temperatures has been evaluated by streaking the microorganisms on NA plates (in triplicates) and by incubating them at different temperatures (4, 40 and 50° C.). After 24 h of incubation, the appearance of colonies on the plates indicates the ability of the strain to grow at a specific temperature. The ability of the bacterial strains to grow at different pH values is determined by streaking the microorganisms on NA plates adjusted at different pH values (5.5, 6.5 and 9.5). Experiment was done in triplicate. After 24 h of incubation, the appearance of bacterial colonies on the plates indicates the ability to grow at a specific pH value.

The salinity tolerance of the strains is determined by streaking the microorganisms on NA plates, modified by the addition of different amount of NaCl (8, 10, 12 and 14%). The experiment is performed in triplicate. After 24 h of incubation, the appearance of colonies on the plates, indicates the ability of the strain to grow at a specific NaCl concentration, thus to tolerate a specific salinity level.

The results are given in Table IV.

TABLE IV

| | Gram | | | Colony | | Growth at | | | | | | | | | |
|---|---|---|---|---|---|---|---|---|---|---|---|---|---|---|---|
| Strain | nature & | | Color of | size | Colony | (° C.) | | | pH | | | Tolerance of NaCl | | | |
| Code | Shape | Motility | colony | (mm) | shape | 4 | 40 | 50 | 5.5 | 6.5 | 9.5 | 8% | 10% | 12% | 14% |
| VMC | Gram | Positive | Cream* | 3-6 | Irregular, | + | + | − | + | + | + | + | − | − | − |
| 10/70 | Positive, | | | | slightly | | | | | | | | | | |
| | rod- | | | | raised and | | | | | | | | | | |
| | shaped | | | | umbonate | | | | | | | | | | |
| | cells | | | | | | | | | | | | | | |
| VMC | Gram | Positive | White* | 2-4 | Pale and | − | + | − | + | + | + | − | − | − | − |
| 10/96 | Positive, | | | | thin, often | | | | | | | | | | |
| | rod- | | | | with | | | | | | | | | | |
| | shaped | | | | amoeboid | | | | | | | | | | |
| | cells | | | | spreading | | | | | | | | | | |

*growth on Nutrient agar

Growth (Fermentability) of Strains for In-Vivo Tests

The biomass production process of the strains is divided into four steps that are: inoculum preparation, fermentation, biomass recovery and biomass drying. Recovery of microbial biomass can be done through several processes such as centrifugation, micro-filtration or ultra-filtration.

Drying process is preferably made by freeze-drying.

Phosphate Solubilization Activity

In-Vitro Assays

Phostate solubilization activity of the strains of the invention was shown by using an in-vitro plate assay according to Pikovskaya method (Yasmin and Bano, 2011).

Tricalcium phosphate (Ca₃(PO₄)₂) was used as inorganic source of phosphorus. The seeding was done superficially using an aliquot (10 μl) of the bacterial suspension (10⁶ CFU/ml).

The following samples were tested:
Paenibacillus polymyxa VMC 10/96
Bacillus simplex VMC 10/70
Bacillus subtilis QST713 (Serenade Max-Bayer)
ATCC 842 (P. polymyxa)

The strains used as specific control in the different tests, were chosen according to what reported on product labels and literature, i.e Bacillus subtilis QST713 (label of Serenade Max-Bayer) because according to Garcia-Lopez and Delgado, 2016 which is reported a typical PGPR/biofertilizer. ATCC842 (P. polymyxa) because, according to (Gaby and Buckley 2012), P. polymyxa promotes plant growth by increasing nutrient availability (fixing nitrogen and solubilizing phosphorus), improving soil porosity, and producing a number of metabolites that promote growth. P. polymyxa ATCC842 can fix atmospheric nitrogen under anaerobic conditions soluble phosphorus is a limiting plant nutrient in soil (Hesham, A. E. and Hashem, M. 2011; Malboobi et al. 2009), and a high proportion of available phosphorus in chemical fertilizers becomes rapidly insoluble, and so unavailable to plants (Malboobi et al. 2009).

Seeded samples were incubated at 30° C. for 7 days and colonies with a clear halo on plate were considered positive for phosphate solubilization.

P-solubilization activity was measured as solubilization index (SI), according to Yasmin and Bano (2011).

For all samples, P-solubilization activity was measured after 7th days and compared one to each other to individuate the best phosphorus solubilizing bacteria (PSB). Solubilization index was determined as follows:

$$S I = \frac{colony diameter + halo zone diameter}{colony diameter}$$

Moreover, the phosphate solubilizing activity of the strains of the invention was evaluated in liquid medium in order to measure the exact quantity of phosphorus liberated from inorganic form (Ca₃(PO₄)₂).

The samples tested were the same as reported above.

The bacteria (2 ml-10⁶ CFU/ml) was inoculated in liquid Pikovskaya medium (pH 6.5) and incubated at 30° C. for 5 days, shaking constantly at 120 rpm.

Samples were then centrifugated at 10,000 rpm for 10 minutes.

An aliquot of the supernatant was taken to measure the soluble phosphorus (P) and the final pH value of the medium.

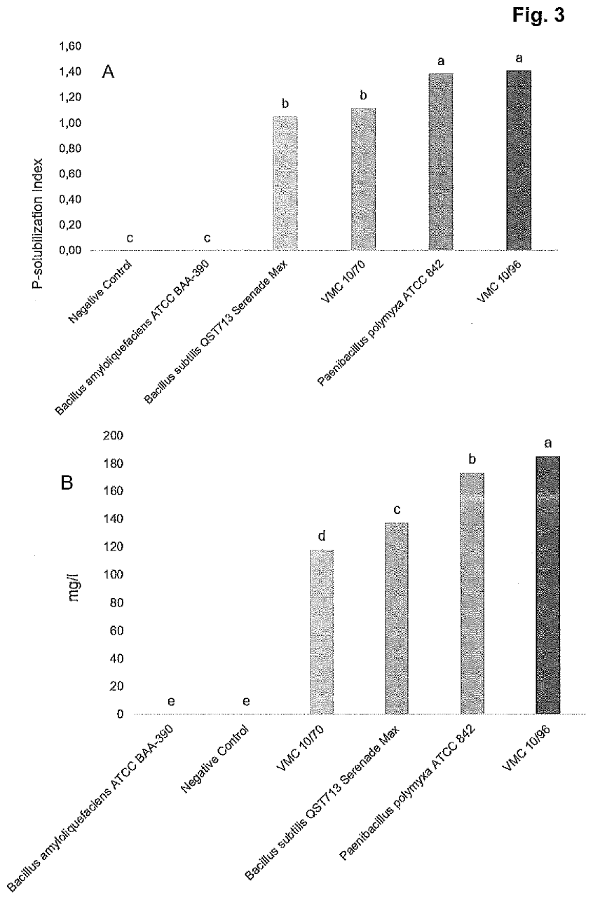

The amount of soluble phosphorus in the supernatant was determined by ion chromatography according to the method: UNI EN ISO 10304-1:2009 comparing the value to a standard curve obtained by measuring a serial dilution of KH₂PO₄ and analyzed by ion chromatography according to the method UNI EN ISO 10304-1:2009. UNI EN ISO 10304-1:2009 is a method used for determination of dissolved anions, such as bromide, chloride, fluoride, nitrate, nitrite, phosphate and sulfate in a liquid sample. The results are summarized in Table V and FIGS. 3 A and B.

TABLE V

| | | | Bacillus | |
|---|---|---|---|---|
| | | | subtilis | |
| | Paenibacillus | Bacillus | QST713 | ATCC 842 |
| | polymyxa | simplex | (Serenade | (P. |
| | VMC 10/96 | VMC 10/70 | Max-Bayer) | polymyxa) |
| Solubi- | 1.41 | 1.12 | 1.05 | 1.39 |
| lization | | | | |
| Index (SI) | | | | |

The solubilization index (SI) of the isolated phosphate solubilizing bacteria ranged from 1.05 to 1.41 at seven days of incubation at 30° C. The results revealed that, among the screened isolates, Paenibacillus polymyxa VMC 10/96 is the most efficient phosphate solubilizer on Picovskaya's plates with SI=1.41 followed by the type strain ATCC 842 (P. polymyxa) with SI=1.39 and Bacillus simplex VMC 10/70 with SI=1.12, whereas the smallest SI of 1.05 was detected from the commercial strain Bacillus subtilis QST713 (Serenade Max-Bayer).

A highly significant (p<0.05) variation of solubilized P concentrations was recorded among the bacterial strains in 5 days of incubation. The results also showed that when the medium was supplemented with (Ca₃(PO₄)₂), the content of soluble phosphate released by the isolates in culture medium increased up to 10 folds in all five bacterial isolates (FIG. 3B). The highest mobilized phosphate value (185 mg/L) was recorded from Paenibacillus polymyxa VMC 10/96 whereas the minimum concentration of soluble-P (118 mg/L) was observed in the cultures of Bacillus simplex VMC 10/70 on day 5 of incubation.

Zinc Solubilization Activity

In-Vitro Assays

In order to determine the zinc-solubilizing capacity of the two bacterial strains used in the present invention, they were screened by a plate assay method using a modified Pikovskaya method (Ghevariya and Desai, 2014). The zinc solubilizing activity was evaluated using zinc oxide (ZnO) as inorganic source of zinc. The isolates were inoculated into agar medium containing 0.1% insoluble zinc (ZnO). The seeding was done superficially using an aliquot (10 μl) of each bacterial suspension, with a concentration of 1.00E+06 CFU/ml. The test organisms were inoculated on these media and incubated at 28° C. for seven days. Zinc solubilization efficiency (SE) was calculated as described by Sharma et al., 2014.

Zinc solubilizing activity was also evaluated in liquid medium in order to measure the exact quantity of zinc liberated from inorganic form ZnO. Each of the strains was inoculated in liquid modified Pikovskaya medium (25 ml), using 250 μl of bacterial culture broth, leading to reach a concentration of 1.00E+6 CFU/ml. After inoculation, tubes were incubated at 28° C. for 3 days, shaking constantly at 120 RPM. The sample was centrifugated at 10,000 rpm for 10 minutes. An aliquot of supernatant was taken to measure soluble Zn and final pH, after filtration with 0.22μ filters. Soluble Zn in the supernatant was measured by ICP-AES (Inductively coupled plasma atomic emission spectroscopy), according to the method: 9.2-9.3 reg. CE 2003/2003 and EN ISO 11885:2009.

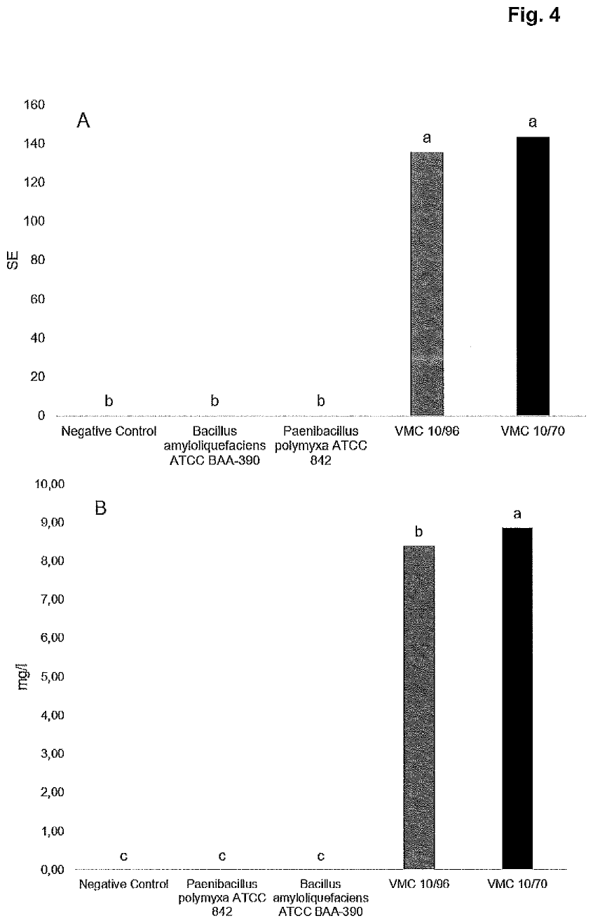

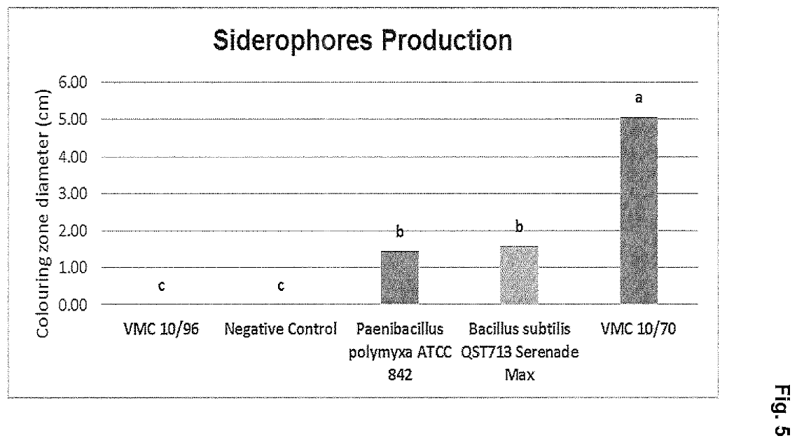

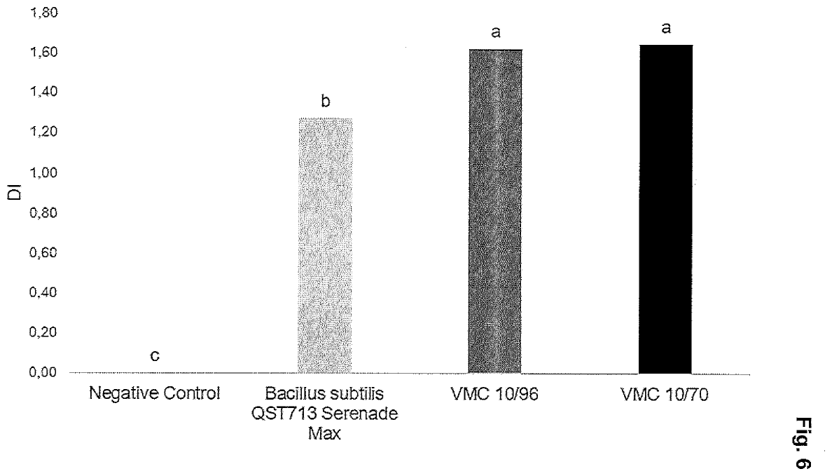

The results summarized in FIG. 4 and they show that the solubilization efficiency (SE) of the isolated zinc solubilizing bacteria ranged from 0 to 143.65 after seven days of incubation at 28° C.

The results revealed that, among the screened isolates, Bacillus simplex VMC 10/70 and Paenibacillus polymyxa VMC 10/96 were the best zinc solubilizer with SE=143.65 and 135.71 respectively, whereas Paenibacillus polymyxa ATCC 842 and Bacillus amyloliquefaciens ATCC BAA-390 were found to be unable to solubilize zinc oxide. A highly significant (p<0.05) variation of solubilized Zn concentrations were recorded among the bacterial strains in 3 days of incubation. The highest dissolved zinc values were recorded from isolate Bacillus simplex VMC 10/70 and Paenibacillus polymyxa VMC 10/96 (8.87 and 8.40 mg/I respectively), whereas the other strains were definitely unable to mobilize zinc from its insoluble form.

Siderophore's Production

In-Vitro Assays

The production of siderophores by Bacillus simplex strain VMC 10/70 was determined according to Schwyn and Neilands (1987) and Miranda et al. (2007). Briefly, 10 μl of bacteria (concentration 1.00E\hat{ }06 CFU/ml) were spotted in triplicate on Nutrient Agar (NA) plates and then incubated at 28° C. for 72 h. After this period, 10 ml of chrome azurol S (CAS) agar medium were poured over the plates. After 24 hours the formation of an orange halo was considered as indicator of siderophore production. The halo was measured according to Omidvari et al. (2010).

In this experiment, the production of organic chelates by one strain was evaluated in an iron-free medium, seeking the induction of siderophore production. The strain was selected for its excellent results obtained on CAS agar medium plates. After evaluating the ability of each strain to release siderophores into culture media, Bacillus simplex strain VMC 10/70 was able to produce the highest amounts of siderophores within 72 hours, among all the bacteria tested, according to the halo diameter (5.1 cm) (FIG. 3).

Endoglucanase Production

In-Vitro Assays

Endoglucanase production by the strains of the invention was measured inoculating each strain into Luria-Bertani agar medium containing 1% CMC (carboxymethylcellulose). The seeding was done superficially using an aliquot (10 μl) of each bacterial suspension (concentration 1.00E+06 CFU/ml). The plates were incubated at 30° C. and after 24 hours the halo size was recorded (Osaka, 2010). CMC degradation ability was measured as the ratio between halo zone diameter and colony diameter (CMC degradation Index).

The results are summarized in FIG. 4 and they show that, after 24 hours of incubation the strains produce the largest amount of Endoglucanase according to the CMC degradation Index (DI). In particular, Bacillus simplex strain is the best endoglucanase producer among all bacteria with a DI=1.65, followed by Paenibacillus polymyxa VMC 10/96 with a DI=1.63, whereas the smallest DI of 1.27 was detected from the commercial strain Bacillus subtilis QST713 (Serenade Max-Bayer).

1-35. (canceled)

36. A microbial consortium comprising
an isolated bacterial strain belonging to the genus Bacillus species simplex characterized by at least one species-specific sequence selected from: SEQ ID NO: 1-4 or sequences having at least 80-99% identity, wherein SEQ ID NO: 1 refers to 16S rRNA gene, SEQ ID NO: 2 refers to gapA gene, SEQ ID NO: 3 refers to uvrA gene and SEQ ID NO: 4 refers to pgk gene, and
an isolated bacterial strain belonging to the genus Paenibacillus, species polymyxa by at least one species-specific sequence selected from: SEQ ID NO: 5-7 or sequences having at least 80-99% identity, wherein SEQ ID NO: 5 refers to 16S rRNA gene, SEQ ID NO: 6 refers to rpoB gene, and SEQ ID NO: 7 refers to nifH gene.

37. The microbial consortium of claim 36, wherein
the isolated bacterial strain belonging to the genus Bacillus species simplex has been deposited at the DSMZ with the accession number DSM32459 and the following name: Bacillus simplex strain VMC10/70, and
the isolated bacterial strain belonging to the genus Paenibacillus, species polymyxa has been deposited at the DSMZ with the accession number DSM32460 and the following name: Paenibacillus polymyxa strain VMC10/96.

38. The microbial consortium of claim 36, wherein the isolated bacterial strains are used in a form selected from the group consisting of: fresh bacteria, frozen bacteria, dry bacteria, lyophilized bacteria, liquid suspension of bacteria, encapsulated bacteria in the form of spores, living bacteria, culture medium, extract of bacteria, supernatant, lysate of bacteria, fraction of bacteria, metabolites derived from said bacteria, and any combination thereof.

39. The microbial consortium according to claim 36, wherein the isolated bacterial strains are mutated and/or edited.

40. The microbial consortium according to claim 36, further comprising microorganisms.

41. The microbial consortium according to claim 40, wherein the microorganisms are bacteria selected the from the group consisting of PGPR or rhizobacteria, yeasts, mycorrhizae, fungi, and derivatives and combinations thereof, wherein:
the PGPR is selected from the group consisting of Aeromonas rivuli, Agromyces fucosus, Bacillus spp. Bacillus mycoides, Bacillus licheniformis, Bacillus subtilis, Bacillus megaterium, Bacillus pumilus, Bacillus safensis, Microbacterium sp., Nocardia globerula, Stenotrophomonas spp., Pseudomonas spp, Pseudomonas fluorescens, Pseudomonas fulva, Pseudoxanthomonas dajeonensis, Rhodococcus coprophilus, Sphingopyxis macrogoltabida, Streptomyces spp., Enterobacter spp., Azotobacter spp., Azospiriullum spp., Rhizobium spp., Herbaspirillum spp., Lactobaccillus spp., Lactobacillus acidophilus, Lactobacillus buchneri, Lactobacillus delbrueckii, Lactobacillus johnsonii, Lactobacillus murinus, Lactobacillus paraplantarum, Lactobacillus pentosus, Lactobacillus plantarum, Lactococcus tactis, and combinations thereof;
the yeast is selected from the group consisting of Candida spp., Candida tropicalis, Saccharomyces spp., Saccharomyces bayanus, Saccharomyces boulardii, Saccharomyces cerevisiae, Saccharomyces exiguous, Saccharomyces pastorianus, Saccharomyces pombe, and combinations thereof;
the mycorrhiza is selected from the group consisting of Glomus spp., Rhizophagus spp., Septoglomus spp., Funneliformis spp., and combinations thereof; and
the fungus is selected from the group consisting of Trichoderma spp., Trichoderma atroviride, Trichoderma viride, Trichoderma afroharzianum, Paecilomyces spp., Beauveria bassiana, Metarhizium spp., Lecanicillium lecanii, Penicillium spp., Aspergillus spp., Conythyrium minitans, Pythium spp, and combinations thereof.

42. The microbial consortium according to claim 36, wherein the strain is used as a fresh or frozen sample or dry, lyophilized or in liquid suspension, or encapsulated in the form of living and/or dead and/or killed cells and/or as spores.

43. The microbial consortium according to any one of claim 40, wherein the ratio between the strain and the further microorganisms ranges from 1:10 to 1:100.

44. The microbial consortium according to claim 36, further comprising a biostimulant.

45. The microbial consortium according to claim 44, wherein the biostimulant is selected from the group consisting of an extract of algae, an extract of microalgae, an extract of plant, a humic acid, a fulvic acid, animal byproducts, or combinations thereof.

46. The microbial consortium according to claim 45, wherein:
the algae are a brown algae selected from the group consisting of Ascophyllum nodosum, Ecklonia maxima, Laminaria saccharina, Laminaria digitata, Fucus spiralis, Fucus serratus, F. vesiculosus, Macrocystis spp., Pelvetia canaliculata, Himantalia elongata, Undaria pinnatifida, Sargassum spp, and combinations thereof;
the microalgae are selected from the group consisting of Spirulina, Scenedesmus, Nannochloropsis, Haematococcus, Chlorella, Phaeodactylum, Arthrospyra, Tetraselmis, Isochrysis, Synechocystis, Clamydomonas, Parietochloris, Desmodesmus, Neochloris, Dunaliella, Thalassiosira, Pavlova, Navicula, Chaetocerous, and combinations thereof; and
the plant is selected from the group consisting of beet, sugar cane, alfalfa, maize, brassica, halophytes, soya, wheat, yucca, quillaja, hop, coffee, citrus, olive, and combinations thereof.

47. A medium, an extract, a supernatant, a lysate, a fraction, or a metabolite obtained/obtainable by culturing the microbial consortium of claim 36, wherein said medium, extract, supernatant, lysate, fraction, or metabolite comprises the cultured bacteria or is a bacteria-free medium.

48. An agricultural composition, comprising the microbial consortium of claim 36, and an agriculturally compatible carrier.

49. An agricultural composition, comprising the medium, extract, supernatant, lysate, fraction or metabolite of claim 47, and an agriculturally compatible carrier.

50. The microbial consortium according to claim 36, formulated as water-soluble concentrates, dispersable concentrates, emulsifiable concentrates, emulsions, suspensions, microemulsion, gel, microcapsules, granules, ultralow volume liquid, wetting powder, dustable powder, or seed coating formulations.

51. The medium, extract, supernatant, lysate, fraction or metabolite according to claim 47, formulated as water-soluble concentrates, dispersable concentrates, emulsifiable concentrates, emulsions, suspensions, microemulsion, gel, microcapsules, granules, ultralow volume liquid, wetting powder, dustable powder, or seed coating formulations.

52. The agricultural composition according to claim 49, formulated as water-soluble concentrates, dispersable concentrates, emulsifiable concentrates, emulsions, suspensions, microemulsion, gel, microcapsules, granules, ultralow volume liquid, wetting powder, dustable powder, or seed coating formulations.

53. The microbial consortium according to claim 42, wherein the strain is the Bacillus simplex strain VMC10/70 and Paenibacillus polymyxa strain VMC10/96.

54. The microbial consortium according to claim 42, wherein the ratio between the strain and further microorganisms is approximately 1:1.

55. The medium, an extract, a supernatant, a lysate, a fraction, or a metabolite of claim 47, wherein the extract is a bacteria-free extract.
